SEQUENCE LISTING (26 sequences)

SEQ ID NO: 1 - DNA, 22 bp, Artificial sequence
  Single strand DNA oligonucleotide
gaaccagaggggagagacagag

SEQ ID NO: 2 - DNA, 22 bp, Artificial sequence
  Single strand DNA oligonucleotide
ccctcagcttgctttttaggag

SEQ ID NO: 3 - DNA, 22 bp, Artificial sequence
  Single strand DNA oligonucleotide
tattggaacatggcctctggat

SEQ ID NO: 4 - DNA, 22 bp, Artificial sequence
  Single strand DNA oligonucleotide
ggtgctgaaggctgattacgtt

SEQ ID NO: 5 - DNA, 20 bp, Artificial sequence
  Single strand DNA oligonucleotide
cttcaagccagaggcctacg

SEQ ID NO: 6 - DNA, 20 bp, Artificial sequence
  Single strand DNA oligonucleotide
ccgcctctgtcttcttcagc

SEQ ID NO: 7 - DNA, 22 bp, Artificial sequence
  Single strand DNA oligonucleotide
gaacaatcccggtgtgtcagga

SEQ ID NO: 8 - DNA, 22 bp, Artificial sequence
  Single strand DNA oligonucleotide
cacccagtggcagccttttaca

SEQ ID NO: 9 - DNA, 22 bp, Artificial sequence
  Single strand DNA oligonucleotide
gtcattgctgaaaccgagaatg

SEQ ID NO: 10 - DNA, 22 bp, Artificial sequence
  Single strand DNA oligonucleotide
gcaaagtactggatgacacgct

SEQ ID NO: 11 - DNA, 22 bp, Artificial sequence
  Single strand DNA oligonucleotide
ggagttatggtgggtatgggtc

SEQ ID NO: 12 - DNA, 22 bp, Artificial sequence
  Single strand DNA oligonucleotide
agtggtgacaaaggagtagcca

SEQ ID NO: 13 - DNA, 20 bp, Artificial sequence
  Single strand DNA oligonucleotide
cgagccgagagtagcagttg

SEQ ID NO: 14 - DNA, 21 bp, Artificial sequence
  Single strand DNA oligonucleotide
cagttggtgatgatgccgtgc

SEQ ID NO: 15 - DNA, 23 bp, Artificial sequence
  Single strand DNA oligonucleotide
ctcatgcccatattccctgcact

SEQ ID NO: 16 - DNA, 24 bp, Artificial sequence
  Single strand DNA oligonucleotide
accaggttggtcttgaactcctgg

SEQ ID NO: 17 - DNA, 20 bp, Artificial sequence
  Single strand DNA oligonucleotide
agccacatcgctcagacacc

SEQ ID NO: 18 - DNA, 20 bp, Artificial sequence
  Single strand DNA oligonucleotide
gtactcagcggccagcatcg

SEQ ID NO: 19 - DNA, 20 bp, Artificial sequence
  Single strand DNA oligonucleotide
cctgggcgttttgttgttgg

SEQ ID NO: 20 - DNA, 20 bp, Artificial sequence
  Single strand DNA oligonucleotide
tgtggctcggtactggcatg

SEQ ID NO: 21 - DNA, 20 bp, Artificial sequence
  Single strand DNA oligonucleotide
agcgaatggaagcccttaca

SEQ ID NO: 22 - DNA, 20 bp, Artificial sequence
  Single strand DNA oligonucleotide
ctcatcgaagtggaccggca

SEQ ID NO: 23 - DNA, 24 bp, Artificial sequence
  Single strand DNA oligonucleotide
gcaataaccgcaatgtgcaatgcc

SEQ ID NO: 24 - DNA, 24 bp, Artificial sequence
  Single strand DNA oligonucleotide
cgccttgtcatgggtgtgcttaaa

SEQ ID NO: 25 - DNA, 26 bp, Artificial sequence
  Single strand DNA oligonucleotide
cctccgaaaccatgaactttctgctc

SEQ ID NO: 26 - DNA, 24 bp, Artificial sequence
  Single strand DNA oligonucleotide
cagcctggctcaccgccttggctt

(19) United States
(12) Patent Application Publication
(10) Pub. No.: US 2009/0169521 A1
(43) Pub. Date: Jul. 2, 2009

(54) Vascularized cardiac tissue and methods of producing and using same

(75) Inventors: Shulamit Levenberg, Moreshet (IL); Lior Gepstein, Haifa (IL); Ayelet Lesman, Haifa (IL); Oren Caspi, Kiryat-Ata (IL)

Correspondence Address: MARTIN D. MOYNIHAN d/b/a PRTSI, INC., P.O. BOX 16446, ARLINGTON, VA, 22215, US

(73) Assignee: Technion Research & Development Foundation Ltd., Haifa (IL)

(21) Appl. No.: 12/003,759

(22) Filed: Dec. 31, 2007

(51) Int. Cl. A61K 45/00; C12N 5/08; A61P 9/10; A61P 9/14

(52) U.S. Cl. 424/93.7; 435/366

(57) ABSTRACT

An isolated composition of matter is disclosed. The composition comprises a heterogeneous population of cells seeded on a scaffold, wherein the heterogeneous population of cells comprises cardiomyocytes, endothelial cells and fibroblast cells. Pharmaceutical compositions comprising same and uses thereof are also disclosed.

### FIELD AND BACKGROUND OF THE INVENTION

The present invention, in some embodiments thereof, relates to a scaffold comprising vascularized cardiac tissue which may be transplanted into the body for the treatment of cardiac disorders.

The adult mammalian heart has limited regenerative capacity and therefore any significant myocardial cell loss is mostly irreversible and may lead to progressive loss of ventricular function and heart failure development. Despite the improvements in several pharmacological, interventional, and surgical therapeutic measures, the prognosis for heart failure patients remains poor. An attractive experimental solution to this significant medical problem may be to repopulate the damaged heart with new myogenic cells. Consequentially, myocardial cell replacement therapy has emerged as a novel experimental therapeutic paradigm aiming to improve the function of the failing heart.

In general, two principal strategies were suggested: the first focused on direct transplantation of isolated cells into the dysfunctional myocardial areas, while the second attempted to combine ex-vivo cells with polymeric scaffolds generating a tissue-engineered muscle construct, followed by in-vivo engraftment of the engineered muscle.

Despite the encouraging results in several animal studies, clinical translation of these approaches have been hampered by the lack of sources for human cardiomyocytes and by the significant cell death following cell transplantation into the hostile ischemic myocardium [Muller-Ehmsen J, et al., Circulation. 2002;105:1720-6]. The latter problem may even be aggravated following the transplantation of clinically-relevant, thick tissue-engineered muscle. Insufficient graft vascularization is considered as one of the main factors responsible for this limited graft survival [Zhang M, et al., J Mol Cell Cardiol. 2001;33:907-21; Miyagawa S, et al., Circulation. 2002;105:2556-2561; Caspi O, Gepstein L. Isr Med Assoc J. 2006;8:208-214]. Although engraftment of myogenic cells within the heart results in an angiogenic reaction, this host-derived graft vascularization [Reffelmann T. et al., J Mol Cell Cardiol. 2003;35:607-613] usually does not provide the transplanted myocytes with the abundant capillary network that normally exists in the heart.

The ability to generate an engineered vascularized skeletal muscle tissue was recently demonstrated by Levenberg S et al., [Nat Biotechnol. 2005;23:879-884].

Osamu Ishii et al., [J Thorac Cardiovasc Surg 2005;130:1358-1363] teach scaffolds comprising cardiomyocytes harvested from neonatal rats.

Birla et al [Tissue Engineering, Vol 11, number 5/6, 2005] teach generation of vascularized cardiac tissue in vivo. The seeded cardiomyocytes were harvested from neonatal rats.

U.S. Pat. No. 6,592,623 teaches electrospun scaffolds comprising cardiac tissue, an extracellular matrix for supporting the cardiac tissue, and a layer of collagen deposited onto the extracellular matrix.

### SUMMARY OF THE INVENTION

According to an aspect of some embodiments of the present invention there is provided an isolated composition of matter comprising a heterogeneous population of cells seeded on a scaffold, wherein the heterogeneous population of cells comprises cardiomyocytes, endothelial cells and fibroblast cells.

According to an aspect of some embodiments of the present invention there is provided an isolated composition of matter comprising a vascularized cardiac tissue attached to a scaffold.

According to an aspect of some embodiments of the present invention there is provided an isolated composition of matter comprising cardiomyocytes seeded on a porous scaffold, wherein a pore of the porous scaffold comprises a minimal average pore diameter of about 200 μm, the scaffold comprising a 50:50 mixture of poly(L-lactic acid) and poly(lactic acid-co-glycolic acid).

According to an aspect of some embodiments of the present invention there is provided a method of ex vivo vascularizing cardiac tissue, the method comprising co-seeding cardiomyocytes and endothelial cells on a scaffold under conditions which allow the formation of at least one 3D endothelial structure within the scaffold, thereby ex vivo vascularizing the cardiac tissue.

According to an aspect of some embodiments of the present invention there is provided a method of treating a cardiac disorder associated with a defective or absent myocardium in a subject, comprising transplanting a therapeutically effective amount of the isolated composition of matter of the present invention into the subject, thereby treating the cardiac disorder associated with a defective or absent myocardium.

According to an aspect of some embodiments of the present invention there is provided a pharmaceutical composition comprising as an active agent the isolated composition of matter of the present invention, and a pharmaceutically acceptable carrier.

According to an aspect of some embodiments of the present invention there is provided a use of the isolated composition of matter of the present invention for the manufacture of a medicament identified for the treatment of a cardiac disorder associated with a defective or absent myocardium.

According to some embodiments of the invention, the heterogeneous population of cells are homogeneously distributed on the scaffold.

According to some embodiments of the invention, the scaffold is a porous scaffold.

According to some embodiments of the invention, the pore of the porous scaffold comprises a minimal average pore diameter of about 200 μm.

According to some embodiments of the invention, the pore of the porous scaffold comprises a maximal average pore diameter of about 800 μm.

According to some embodiments of the invention, the scaffold comprises poly(L-lactic acid) and poly(lactic acid-co-glycolic acid).

According to some embodiments of the invention, the scaffold comprises a 50:50 mixture of poly(L-lactic acid) and poly(lactic acid-co-glycolic acid).

According to some embodiments of the invention, the scaffold is biodegradable.

According to some embodiments of the invention, the scaffold is non-biodegradable.

According to some embodiments of the invention, the scaffold comprises a material selected from the group consisting of collagen-GAG, collagen, fibrin, PLA, PGA, PLA-PGA co-polymer, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, non-biodegradable polyurethane, polyurea, poly(ethylene vinyl acetate), polypropylene, polymethacrylate, polyethylene, polycarbonate and poly(ethylene oxide).

According to some embodiments of the invention, the scaffold is coated with a gel.

According to some embodiments of the invention, the gel is selected from the group consisting of a collagen gel, an alginate, an agar, a growth factor-reduced Matrigel, and MATRIGEL™.

According to some embodiments of the invention, the fibroblasts are human embryonic fibroblasts.

According to some embodiments of the invention, the cardiomyocytes are human cardiomyocytes.

According to some embodiments of the invention, the cardiomyocytes are derived from stem cells.

According to some embodiments of the invention, the stem cells are embryonic stem cells.

According to some embodiments of the invention, the stem cells are adult stem cells.

According to some embodiments of the invention, the endothelial cells are embryonic stem cell-derived endothelial cells or umbilical vein endothelial cells.

According to some embodiments of the invention, the embryonic stem cell-derived endothelial cells are mammalian stem cell derived endothelial cells.

According to some embodiments of the invention, the mammalian embryonic stem cell-derived endothelial cells are human stem cell derived endothelial cells.

According to some embodiments of the invention, the endothelial cells form part of a 3D endothelial structure.

According to some embodiments of the invention, the 3D endothelial structure comprises a lumen.

According to some embodiments of the invention, the scaffold is a non-fibrous scaffold.

According to some embodiments of the invention, the vasculature of the vascularized cardiac tissue is sufficient for survival for at least two weeks of the cardiac tissue following transplantation.

According to some embodiments of the invention, the vascularized cardiac tissue comprises fibroblasts.

According to some embodiments of the invention, the vascularized cardiac tissue comprises endothelial cells.

According to some embodiments of the invention, the vascularized cardiac tissue comprises smooth muscle cells.

According to some embodiments of the invention, the vasculature of the vascularized cardiac tissue forms part of a 3D structure.

According to some embodiments of the invention, the 3D structure comprises a lumen.

According to some embodiments of the invention, the percent of the vasculature of the vascularized cardiac tissue is at least 1%.

According to some embodiments of the invention, the cardiac tissue is capable of spontaneous contraction.

According to some embodiments of the invention, the cardiac tissue is capable of responding to a chronotropic agent in a similar fashion to non-engineered cardiac tissue.

According to some embodiments of the invention, the scaffold further comprises endothelial cells seeded on the porous scaffold.

According to some embodiments of the invention, the scaffold further comprises fibroblasts seeded on the porous scaffold.

According to some embodiments of the invention, the scaffold further comprises smooth muscle cells seeded on the porous scaffold.

According to some embodiments of the invention, the conditions comprise an ex vivo culturing period of at least 1 week.

According to some embodiments of the invention, the conditions comprise co-seeding fibroblast cells with the cardiomyocytes and the endothelial cells on the scaffold.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are photographs illustrating immunohistochemistry of the engineered human cardiac tissue. The scaffolds were stained with anti-troponin-I and anti-sarcomeric-α-actinin antibodies (cardiac-specific markers) and with anti-CD-31 antibodies (endothelial marker) (DAB-brown). (FIG. 1A) Scaffolds containing only hESC-CM (FIG. 1B) Scaffolds containing hESC-CM+HUVEC. (FIG. 1C) Scaffolds containing hESC-CM+HUVEC+EmF. Note the high density of vascularization formed in the tri-culture scaffolds.

FIGS. 2A-C are photographs of immunofluorescent staining of the engineered cardiac tissue. Co-staining using anti-troponin I (red) and anti-vWF (green) antibodies (left panel), and using anti-CD-31 (red) and anti-vWF (green) antibodies (right panel). Nuceli are shown in blue. Shown are scaffolds containing only hESC-CM (FIG. 2A), hESC-CM+HUVEC (FIG. 2B), and hESC-CM+HUVEC+EmF (FIG. 2C). Note that the addition of EmF to the constructs containing hESC-CM and EC resulted in the generation of structurally-organized vascularized engineered cardiac muscle (FIG. 2C).

FIGS. 3A-E are photographs illustrating the vascularization of the human cardiac tissue. Immunohistochemistry using anti-CD-31 antibodies (brown) (left panel) and immunofluorescent staining with anti-CD-31 (red) and anti-vWF (green) antibodies. Nuceli are shown in blue. (FIG. 3A) Scaffolds containing co-cultures of hESC-CM+HUVEC. (FIGS. 3B-C) Scaffolds containing the tri-culture of hESC-CM+HUVEC+EmF with a HUVEC/EmF ratio of 2:1 (FIG. 3B) or 1:1 (FIG. 3C). (FIG. 3D) Generation of a vascularized human cardiac tissue also when using hESC-EC. (FIG. 3E) Differentiation of EmF to smooth muscle cells as indicated by co-immunostaining for α-smooth-muscle-actin (red), vWF (green), and Dapi (blue). The SMA⁺ cells integrated into the formed vessel and are localized adjacent to vWF+ cells.

FIGS. 4A-D are bar graphs and photograph illustrating the results of a quantitative analysis of the vascularization process. The analysis was based on immunofluorescent staining using anti-vWF antibody. The parameters that were investigated were: (FIG. 4A) the number of lumens per mm², (FIG. 4B) the lumen area density, and (FIG. 4C) the EC area density. In the tri-culture constructs high vessel network was created (lumens/mm²: 5.12±2, lumen area density: 1.25±0.45 and endothelial density: 1.57±0.51%) in contrast to the co-culture (0.08±0.08, 0.01±0.01 and 0.16±0.06 respectively). (p=0.01 (Number of lumens/mm²), p=0.02 (Lumen area density), p=0.058 (EC density)) In the constructs containing only hESC-CM almost no vascularization was noted (EC density: 0.08±0.02%) (not shown) (FIG. 4D) RT-PCR analysis for the expression of PDGF-B, VEGF, Ang1, and bFGF in the three groups. Increased expression of VEGF, PDGF-B and Ang1 was revealed in the tri-culture cardiac tissue upon addition of the EmF.

FIGS. 5A-D are bar graphs and photographs of results following temporal assessment of endothelial cell viability and proliferation. (FIGS. 5A, B) Endothelial cell viability was evaluated at several time points following cell seeding (1 hr, 24 hrs, 72 hrs, and 1 wk) in the presence and in the absence of EmF using Calcien-AM (Green-staining viable cells) and Ethidium Homodimer-1 (Red-staining dead cells). (Picture in panel 5A—viability assessed following 1 week.) (FIG. 5C) Double immunofluorescent staining using anti-human-Ki67 (red) and anti-vWF (green) antibodies for the identification of proliferating endothelial cells (pointed by arrows). (FIG. 5D) Quantitative analysis of EC proliferation. In the tri-culture constructs the percentage of cycling EC was significantly higher (10.7±1%) than in the co-culture (4.4±1.5%), (p<0.01).

FIGS. 6A-D illustrate cardiomyocyte proliferation and RT-PCR analysis. (FIG. 6A) Double immunostaining using anti-troponin-I (red), anti-human-ki-67 (green) antibodies (right) and with Dapi (blue) for nuclear staining (left). Most of the proliferating cells (pointed by arrow) were those with higher nuclear to cytoplasmatic ratio and limited sarcomeric organization. (FIG. 6B) Quantitative analysis of cardiomyocytes proliferation in the three groups. The presence of EC in the engineered cardiac tissue significantly increased the rate of proliferating cardiomyocytes, both in the presence (30.5±6.9%) and absence (24.6±5.1%) of EmF, when compared with scaffolds containing only cardiomyocytes (8.2±1.4%) (p=0.012 and p=0.048, respectively). (FIG. 6C) RT-PCR analysis evaluating both markers of early, immature cardiomyocytes (ANF, NKX 2.5, MEF2C and a-skeletal actin) and markers of more mature and differentiated cardiomyocytes (MLC-2V, a-MHC, a-cardiac actin). (FIG. 6D) Quantitative Real time RT-PCR analysis indicating both up regulation of mature cardiomyocytes genes (MHC and Troponin I) and early, immature cardiomyocytes genes (nkx2.5 and ANF) in the triculture scaffolds. (*) p<0.05.

FIGS. 7A-F are photographs illustrating ultrastructural characterization of the engineered cardiac tissue. Transmission electron microscopy demonstrating the presence of both cardiomyocytes in early and more mature stages of development. (FIG. 7A) Early developing cells showing disorganized myofibrils that in some cases were associated with a distinct electron dense material (Z-bodies). (FIG. 7B) Relatively mature cells showing myofibrils organized in similar direction and confined to parallel Z bands (Zb) forming a typical sarcomeric pattern. Beyond the presence of mitochondria (Mi), the existence of T-tubules (TT) and sarcoplasmic reticulum (SR) indicates a more developed maturation stage. (FIGS. 7C, D) Presence of the specialized cell-cell junctions including the intercalated disc containing desmosomes (FIG. 7D) and gap junctions (GJ). (Figures E,F) Double immunofluorescent staining using anti Troponin I and Cox 43 antibodies revealed the presence of gap junctions composed of connexin 43.

FIGS. 8A-B are images of impulse propagation during Laser-confocal Ca⁺² imaging studies utilizing the free Ca⁺² binding dye, Fluo-4. (FIG. 8A) Line scan images demonstrated synchronous surges of intracellular Ca⁺² levels within the contracting hESC-CM. (FIG. 8B) In the presence of 1-heptanol, a gap-junction uncoupler, impulse propagation was completely inhibited.

FIGS. 9A-B are images of H&E staining demonstrating that the engineered cardiac scaffold is attached to the rat myocardium. (FIG. 9A) Low magnification, the scaffold area is indicated by arrow. (FIG. 9B) Higher magnification, the scaffold substance (indicated by arrows) is present and the graft is tightly attached to the host myocardium.

FIGS. 10A-B are low magnification (10A) and high magnification (10B) images of H&E staining demonstrating functional blood vessels in the scaffold area. Arrow indicates the scaffold substance and arrows heads indicate red blood cells within the vessels.

FIGS. 11A-D are images of blood vessel immunostaining. The formation of human and rat-derived vasculature within the scaffold was confirmed by immunostaining using both anti-human-specific CD31 (FIGS. 11A, B in red) and anti-smooth muscle actin (FIGS. 11C, D in red, staining both rat and human vessels) antibodies.

FIGS. 12A-D are bar graphs illustrating blood vessel quantitative assessment. (FIGS. 12A, B) Quantitative assessment of number of vessels per square mm of graft area in the two groups; tri-culture cell combination (CHM) and cardiac cells only (Cardio) constructs. (FIG. 12A) Rat and human derived vessels were measured by staining with smooth muscle actin antibody (SMA). (FIG. 12B) human derived vessels were identified by staining with CD31 antibody. The vessels area distribution (see legend attached, area in microns) is appeared on the graphs. (FIGS. 12C, D) Quantitative assessment of endothelial cells percent (left) and lumen area density (right) of graft area.

FIGS. 13A-D are photographs of immunostained scaffolds following in vivo transplantation illustrating perfusion of the scaffold. Functional vessels were identified in the scaffold area by injection of Lectin-HPA (FIG. 13A, B) and fluorescence Microspheres (FIG. 13C, D). Anastomosis between human vessels and host derived vasculature was confirmed by immunostaining using human specific endothelial marker CD31 antibody and the presence of Microspheres in those CD31⁺ vessels (FIG. 13D).

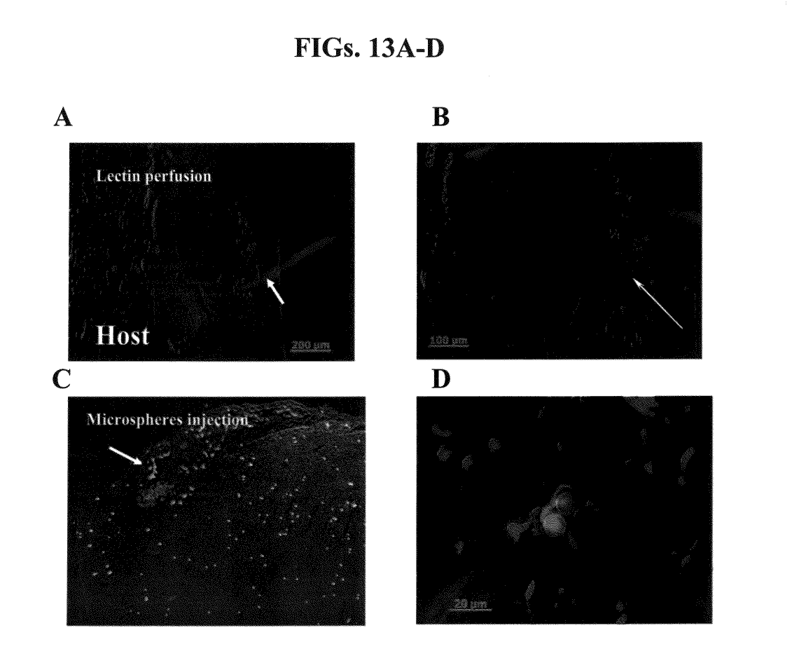

FIGS. 14A-F are photographs of immunostained scaffolds following in vivo transplantation illustrating the presence of the human cardiomyocytes within the graft area. (FIGS. 14A-C) hES-CM appeared to be elongated and well-aligned with each other as indicated by anti Troponin I antibody (red). (FIG. 14D) Electromechanical integration of the cardiomyocytes within the scaffold was suggested by positive immunostaining for connexin-43 (green dots). (FIG. 14E, F) The cardiomyocytes were pre-labeled with DiO (green) and stained with anti-Troponin I antibody (red) to insure their existence in the graft after two weeks in-vivo.

### DESCRIPTION OF EMBODIMENTS OF THE INVENTION

The present invention, in some embodiments thereof, relates to a scaffold comprising vascularized cardiac tissue which may be transplanted into the body for the treatment of cardiac disorders.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Treatments for chronic heart failure include medical management with pharmaceutical drugs, diet and exercise, and mechanical assist devices, which are costly and risk failure and infection. The results of all these effort are disappointing with a 75% five year mortality rate for heart failure victims. Thus, the landscape for cardiac treatment is turning in recent years to transplantation of tissue or cells. However, as of yet, these transplants have only met with a limited success rate. It has been suggested that a primary reason for non-immune cardiac tissue transplantation failure may be the result of angiogenic inefficiency. Cardiac tissue is particularly sensitive to this phenomenon, mainly due to its high metabolic demands, its inherent intolerance of anaerobic metabolism and the compact nature of the cardiac muscle strands within.

Thus, construction of clinically-relevant cardiac tissues must allow full thickness perfusion of the preformed cardiac muscle. This issue is even of greater importance when considering the scarce vascularization of the myocardial scar.

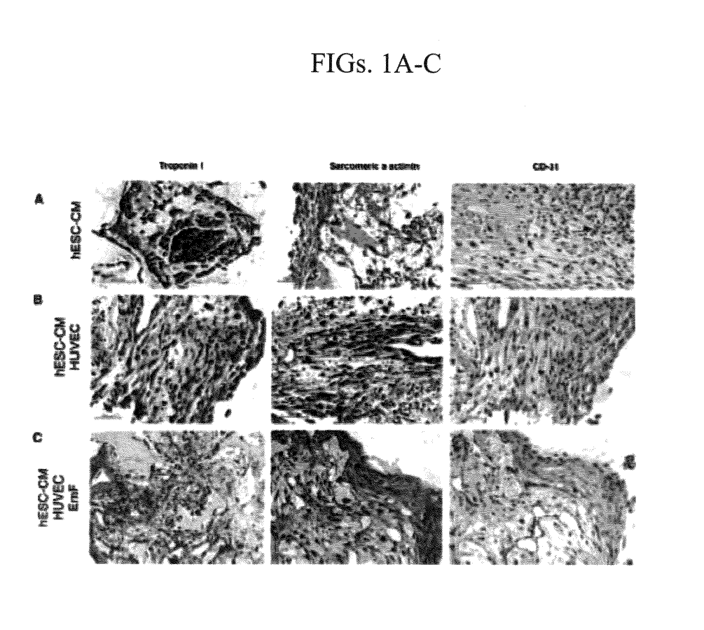

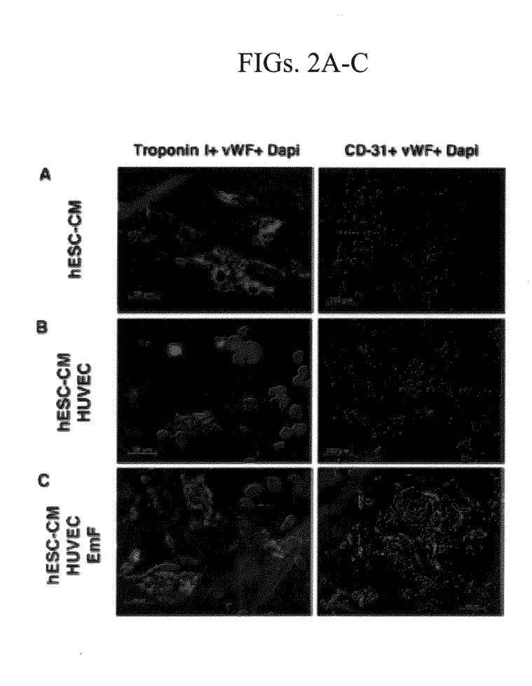

Whilst reducing the present invention to practice, the present inventors have discovered that under appropriate conditions, the seeding of cardiomyocytes (derived from human embryonic stem cells) on a scaffold in the presence of endothelial cells promotes establishment of 3D endothelial tube structures (FIG. 1B). The presence of fibroblast cells was shown to increase the vascularization therein (FIGS. 1C and 2C).

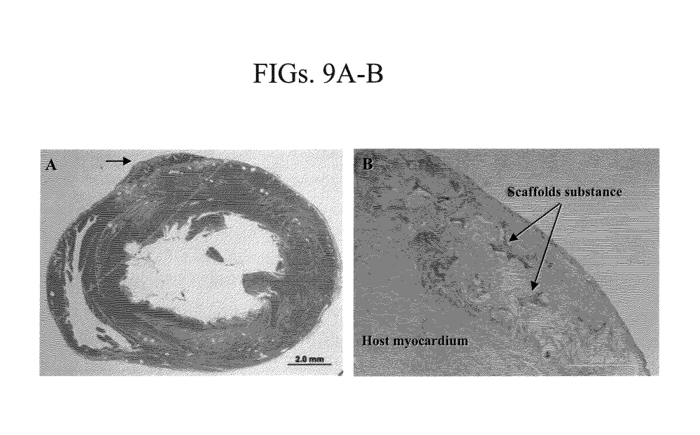

Furthermore, the present inventors have shown that scaffold-seeded cardiomyocytes and endothelial cells promote functional vascularization. This is evidenced by the data showing that scaffold-seeded cardiomyocytes in the presence of endothelial cells and fibroblast cells, are capable of surviving for at least two weeks following transplantion into a healthy rat (FIGS. 9A-B-14A-F).

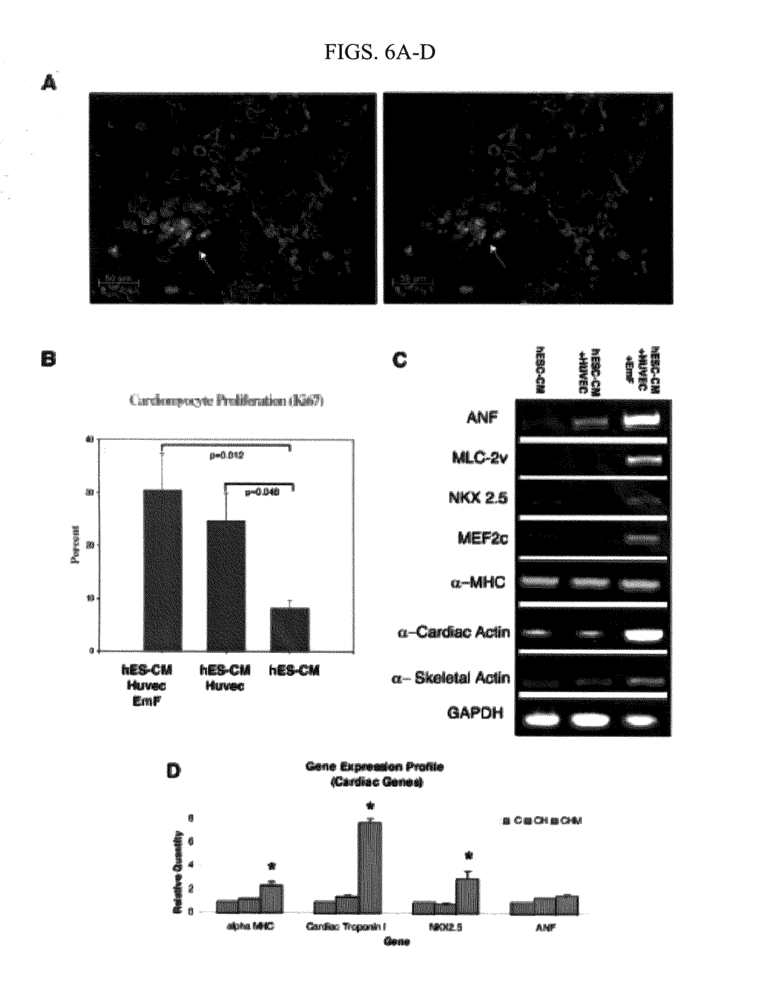

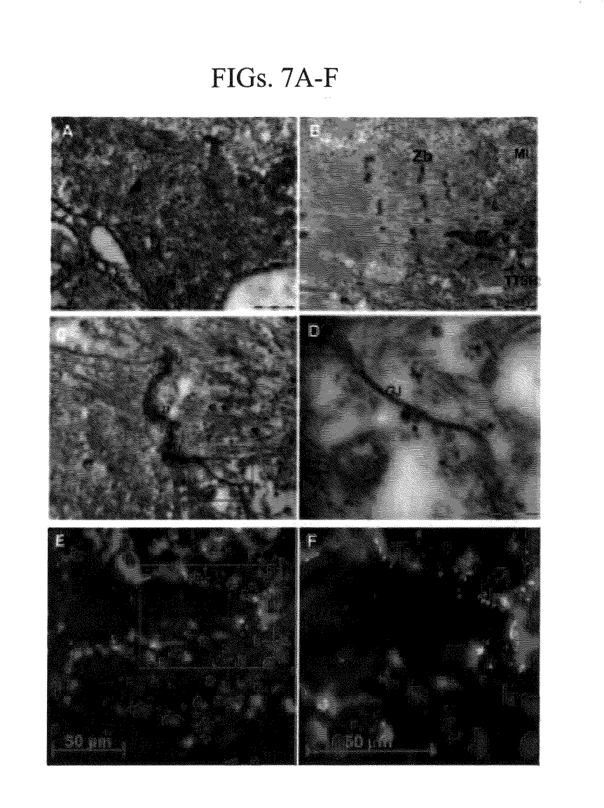

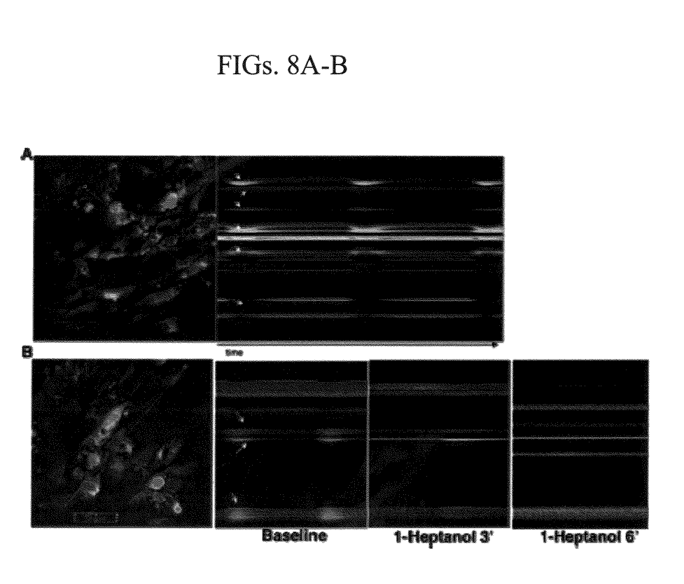

Cardiac tissue generated according to some embodiments of the present invention was shown to express markers of mature differentiated cardiomyocytes (Myosin light chain 2V, α myosin light chain, α-cardiac actin and Troponin I) as measured by RT-PCR (FIGS. 6C-D). In addition, the cardiac tissue was shown to comprise gap junctions (FIGS. 7C-D) and to be capable of spontaneous synchronous contractions (FIG. 8A). Furthermore, the engineered cardiac tissue of the present invention was shown to be capable of responding to chronotropic agents (such as B agonists and muscarinic agonists) in a similar fashion to non-engineered cardiac tissue.

Thus, according to one aspect of the present invention, there is provided a method of ex vivo vascularizing a cardiac tissue, the method comprising co-seeding cardiomyocytes and endothelial cells on a scaffold under conditions which allow the formation of at least one 3D endothelial structure within the scaffold, thereby ex vivo vascularizing the cardiac tissue.

As used herein, the phrase “vascularizing a cardiac tissue” refers to formation of at least a part of a 3D blood vessel network in the cardiac tissue. Typically, the blood vessel network is comprised of endothelial cells. The vasculature may be at any stage of formation as long as it comprises at least one 3D endothelial structure. Examples of 3D endothelial structures include, but are not limited to tube-like structures, such as those comprising a lumen.

The term “cardiac tissue” as used herein refers to a population of cells that together are able to function to fulfill at least one functional phenotype specific to cardiac tissue (e.g. appropriate response to a chronotropic agent and/or ability to spontaneously contract in a synchronized function). The cardiac tissue is typically a mammalian cardiac tissue and more preferably a human cardiac tissue.

As mentioned herein above, the method of the present invention is effected by co-seeding cardiomyocytes and endothelial cells on a scaffold.

The term “co-seeding” as used herein refers to a seeding of both the cardiomyocytes and endothelial cells at substantially the same time. According to one embodiment the cardiomyocytes and endothelial cells are initially pooled to generate a mixture of cardiomyocytes and endothelial cells and then seeded onto the scaffold. According to this embodiment, at the seeding stage, the cardiomyocytes and endothelial cells are homogeneously distributed on the scaffold.

According to another embodiment one of the two cell types described herein above is seeded first and immediately the second cell type is then seeded onto the scaffold.

As used herein, the term “cardiomyocytes” refers to fully or at least partially differentiated cardiomyocytes. Thus, cardiomyocytes may be derived from stem cells (such as embryonic stem cells or adult stem cells, such as mesenchymal stem cells). Methods of differentiating stem cells along a cardiac lineage are well known in the art—[Muller-Ehmsen J, et al., Circulation. 2002;105:1720-6; Zhang M, et al., J Mol Cell Cardiol. 2001;33:907-21, Xu et al, Circ Res. 2002;91:501-508, and U.S. Pat. Appl. No. 20050037489, the contents of which are incorporated by reference herein]. According to one embodiment the stem cells are derived from human stem cell lines, such as H9.2 (Amit, M. et al., 2000. Dev Biol. 227:271).

According to one embodiment the cardiomyocytes of the present invention are at least capable of spontaneous contraction. According to another embodiment, the cardiomyocytes of the present invention express at least one marker (more preferably at least two markers and even more preferably at least three markers) of early-immature cardiomyocytes (e.g. atrial natriuretic factor (ANF), Nkx2.5, MEF2C and α-skeletal actin). According to another embodiment, the cardiomyocytes of the present invention express at least one marker (more preferably at least two markers and even more preferably at least three markers) of fully differentiated cardiomyocytes (e.g. MLC-2V, α-MHC, α-cardiac actin and Troponin I).

Screening of partially differentiated cardiomyocytes may be performed by a method enabling detection of at least one characteristic associated with a cardiac phenotype, as described hereinbelow, for example via detection of cardiac specific mechanical contraction, detection of cardiac specific structures, detection of cardiac specific proteins, detection of cardiac specific RNAs, detection of cardiac specific electrical activity, and detection of cardiac specific changes in the intracellular concentration of a physiological ion.

Various techniques can be used to detect each of cardiac specific mechanical contraction, cardiac specific structures, cardiac specific proteins, cardiac specific RNAs, cardiac specific electrical activity, and cardiac specific changes in the intracellular concentration of a physiological ion. For example, detection of cardiac specific mechanical contraction may be effected visually using an optical microscope. Alternately, such detection can be effected and recorded using a microscope equipped with a suitable automated motion detection system. Detection of cardiac specific structures may be performed via light microscopy, fluorescence affinity labeling and fluorescence microscopy, or electron microscopy, depending on the type of structure whose detection is desired. Detection of cardiac specific proteins may be effected via fluorescence affinity labeling and fluorescence microscopy. Alternately, techniques such as Western immunoblotting or hybridization micro arrays (“protein chips”) may be employed. Detection of cardiac specific RNAs is preferably effected using RT-PCR. Alternately, other commonly used methods, such as hybridization microarray (“RNA chip”) or Northern blotting, may be employed. RT-PCR can be used to detect cardiac specific RNAs. Detection of cardiac specific changes in the intracellular concentration of a physiological ion, such as calcium, is preferably effected using assays based on fluorescent ion binding dyes such as the fura-2 calcium binding dye (for example, refer to Brixius, K. et al., 1997. J Appl Physiol. 83:652). Such assays can be advantageously used to detect changes in the intracellular concentration of calcium ions, such as calcium transients. Detection of cardiac specific electrical activity of the cells may be effected by monitoring the electrical activity thereof via a multielectrode array. Suitable multielectrode arrays may be obtained from Multi Channel Systems, Reutlingen, Germany. To detect cardiac specific electrical activity in the partially differentiated cells, the latter can be advantageously cultured, under conditions suitable for inducing cardiac differentiation directly on a multielectrode array, thereby conveniently enabling monitoring the electrical activity of such cells and tissues. Regions of embryoid bodies displaying cardiac differentiation, preferably in the form of cardiac specific mechanical contraction, can be advantageously microdissected from embryoid bodies and cultured on microelectrode arrays

As mentioned, the method of the present invention is effected by seeding both cardiomyocytes and endothelial cells on a scaffold. The endothelial cells may be human embryonic stem cell (hESC)-derived endothelial cells (Levenberg, et al., Proc Natl Acad Sci USA (2002) 99, 4391-4396, the contents of which are incorporated by reference herein), or primary endothelial cells cultured from e.g. human umbilical vein (HUVEC), or biopsy-derived endothelial cells such as from the aorta or umbilical artery. The endothelial cells of the present invention may also be derived from humans (either autologous or non-autologous) e.g. from the blood or bone marrow. In addition the endothelial cells may be derived from other mammals, for example, humans, mice or cows. For example, endothelial cells may be retrieved from bovine aortic tissue.

In one embodiment, human embryonic endothelial cells are produced by culturing human embryonic stem cells in the absence of LIF and bFGF to stimulate formation of embryonic bodies, and isolating PECAM1 positive cells from the population. HUVEC may be isolated from tissue according to methods known to those skilled in the art or purchased from cell culture laboratories such as Cambrex Biosciences or Cell Essentials.

As used herein, the term “scaffold” refers to a 3 dimensional matrix upon which cells may be cultured (i.e., survive and preferably proliferate for a predetermined time period).

The scaffold of the present invention may be made uniformly of a single polymer, co-polymer or blend thereof. However, it is also possible to form a scaffold according to the invention of a plurality of different polymers. There are no particular limitations to the number or arrangement of polymers used in forming the scaffold. According to one embodiment the polymer scaffold is made of non-fibrous. According to another embodiment, the scaffold is not electrospun.

Both the choice of polymer and the ratio of polymers in a co-polymer may be adjusted to optimize the stiffness of the scaffold. The molecular weight and cross-link density of the scaffold may also be regulated to control both the mechanical properties of the scaffold and the degradation rate (for degradable scaffolds). The mechanical properties may also be optimized to mimic those of the tissue at the implant site. The shape and size of the final scaffold should be adapted for the implant site and tissue type.

Scaffold material may comprise natural or synthetic organic polymers that can be gelled, or polymerized or solidified (e.g., by aggregation, coagulation, hydrophobic interactions, or cross-linking) into a 3-D open-lattice structure that entraps water or other molecules, e.g., to form a hydrogel. Structural scaffold materials may comprise a single polymer or a mixture of two or more polymers in a single composition. Additionally, two or more structural scaffold materials may be co-deposited so as to form a polymeric mixture at the site of deposition. Polymers used in scaffold material compositions may be biocompatible, biodegradable and/or bioerodible and may act as adhesive substrates for cells. In exemplary embodiments, structural scaffold materials are easy to process into complex shapes and have a rigidity and mechanical strength suitable to maintain the desired shape under in vivo conditions.

In certain embodiments, the structural scaffold materials may be non-resorbing or non-biodegradable polymers or materials.

The phrase “non-biodegradable polymer”, as used herein, refers to a polymer or polymers which at least substantially (i.e. more than 50%) do not degrade or erode in vivo. The terms “non-biodegradable” and “non-resorbing” are equivalent and are used interchangeably herein.

Such non-resorbing scaffold materials may be used to fabricate materials which are designed for long term or permanent implantation into a host organism. In exemplary embodiments, non-biodegradable structural scaffold materials may be biocompatible. Examples of biocompatible non-biodegradable polymers which are useful as scaffold materials include, but are not limited to, polyethylenes, polyvinyl chlorides, polyamides such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates such as ethylene vinyl acetate copolymer, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art.

In other embodiments, the structural scaffold materials may be a “bioerodible” or “biodegradable” polymer or material.

The phrase “biodegradable polymer” as used herein, refers to a polymer or polymers which degrade in vivo, and wherein erosion of the polymer or polymers over time occurs concurrent with or subsequent to release of the cardiac tissue. The terms “biodegradable” and “bioerodible” are equivalent and are used interchangeably herein.

Such bioerodible or biodegradable scaffold materials may be used to fabricate temporary structures. In exemplary embodiments, biodegradable or bioerodible structural scaffold materials may be biocompatible. Examples of biocompatible biodegradable polymers which are useful as scaffold materials include, but are not limited to, polylactic acid, polyglycolic acid, polycaprolactone, and copolymers thereof, polyesters such as polyglycolides, polyanhydrides, polyacrylates, polyalkyl cyanoacrylates such as n-butyl cyanoacrylate and isopropyl cyanoacrylate, polyacrylamides, polyorthoesters, polyphosphazenes, polypeptides, polyurethanes, polystyrenes, polystyrene sulfonic acid, polystyrene carboxylic acid, polyalkylene oxides, alginates, agaroses, dextrins, dextrans, polyanhydrides, biopolymers such as collagens and elastin, alginates, chitosans, glycosaminoglycans, and mixtures of such polymers. In still other embodiments, a mixture of non-biodegradable and bioerodible and/or biodegradable scaffold materials may be used to form a biomimetic structure of which part is permanent and part is temporary.

PLA, PGA and PLA/PGA copolymers are particularly useful for forming the scaffolds of the present invention. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. The erosion of the polyester scaffold is related to the molecular weights. The higher molecular weights, weight average molecular weights of 90,000 or higher, result in polymer scaffolds which retain their structural integrity for longer periods of time; while lower molecular weights, weight average molecular weights of 30,000 or less, result in both slower release and shorter scaffold lives. For example, poly(lactide-co-glycolide) (50:50) degrades in about six weeks following implantation.

According to a preferred embodiment of this aspect of the present invention the scaffold comprises a 50:50 mixture of poly(L-lactic acid) and poly(lactic acid-co-glycolic acid.

In certain embodiments, the structural scaffold material composition is solidified or set upon exposure to a certain temperature; by interaction with ions, e.g., copper, calcium, aluminum, magnesium, strontium, barium, tin, and di-, tri- or tetra-functional organic cations, low molecular weight dicarboxylate ions, sulfate ions, and carbonate ions; upon a change in pH; or upon exposure to radiation, e.g., ultraviolet or visible light. In an exemplary embodiment, the structural scaffold material is set or solidified upon exposure to the body temperature of a mammal, e.g., a human being. The scaffold material composition can be further stabilized by cross-linking with a polyion.

In an exemplary embodiment, scaffold materials may comprise naturally occurring substances, such as, fibrinogen, fibrin, thrombin, chitosan, collagen, alginate, poly(N-isopropylacrylamide), hyaluronate, albumin, collagen, synthetic polyamino acids, prolamines, polysaccharides such as alginate, heparin, and other naturally occurring biodegradable polymers of sugar units.

In certain embodiments, structural scaffold materials may be ionic hydrogels, for example, ionic polysaccharides, such as alginates or chitosan. Ionic hydrogels may be produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix. In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole). Polyphosphazenes are polymers with backbones consisting of nitrogen and phosphorous atoms separated by alternating single and double bonds. Each phosphorous atom is covalently bonded to two side chains. Polyphosphazenes that can be used have a majority of side chains that are acidic and capable of forming salt bridges with di- or trivalent cations. Examples of acidic side chains are carboxylic acid groups and sulfonic acid groups. Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol, and glucosyl. Bioerodible or biodegradable polymers, i.e., polymers that dissolve or degrade within a period that is acceptable in the desired application (usually in vivo therapy), will degrade in less than about five years or in less than about one year, once exposed to a physiological solution of pH 6-8 having a temperature of between about 25° C. and 38° C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Typically, the scaffolds of the present invention are porous. The porosity of the scaffold may be controlled by a variety of techniques known to those skilled in the art. The minimum pore size and degree of porosity is dictated by the need to provide enough room for the cells and for nutrients to filter through the scaffold to the cells. The maximum pore size and porosity is limited by the ability of the scaffold to maintain its mechanical stability after seeding. As the porosity is increased, use of polymers having a higher modulus, addition of stiffer polymers as a co-polymer or mixture, or an increase in the cross-link density of the polymer may all be used to increase the stability of the scaffold with respect to cellular contraction.

According to a preferred embodiment of this aspect of the present invention, the scaffold has a minimal average pore diameter of about 200 μm and a maximal average pore diameter of about 800 μm.

The scaffolds may be made by any of a variety of techniques known to those skilled in the art. Salt-leaching, porogens, solid-liquid phase separation (sometimes termed freeze-drying), and phase inversion fabrication may all be used to produce porous scaffolds. Fiber pulling and weaving (see, e.g. Vacanti, et al., (1988) Journal of Pediatric Surgery, 23: 3-9) may be used to produce scaffolds having more aligned polymer threads. Those skilled in the art will recognize that standard polymer processing techniques may be exploited to create polymer scaffolds having a variety of porosities and microstructures.

Scaffold materials are readily available to one of ordinary skill in the art, usually in the form of a solution (suppliers are, for example, BDH, United Kingdom, and Pronova Biomedical Technology a.s. Norway). For a general overview of the selection and preparation of scaffolding materials, see the American National Standards Institute publication No. F2064-00 entitled Standard Guide for Characterization and Testing of Alginates as Starting Materials Intended for Use in Biomedical and Tissue Engineering Medical Products Applications”.

Therapeutic compounds or agents that modify cellular activity can also be incorporated (e.g. attached to, coated on, embedded or impregnated) into the scaffold material. Campbell et al (US Patent Application No. 20030125410) which is incorporated by reference as if fully set forth by reference herein, discloses methods for fabrication of 3D scaffolds for stem cell growth, the scaffolds having preformed gradients of therapeutic compounds. The scaffold materials, according to Campbell et al, fall within the category of “bio-inks”. Such “bio-inks” are suitable for use with the compositions and methods of the present invention.

Exemplary agents that may be incorporated into the scaffold of the present invention include, but are not limited to those that promote cell adhesion (e.g. fibronectin, integrins), cell colonization, cell proliferation, cell differentiation, cell extravasation and/or cell migration. Thus, for example, the agent may be an amino acid, a small molecule chemical, a peptide, a polypeptide, a protein, a DNA, an RNA, a lipid and/or a proteoglycan.

Proteins that may be incorporated into the scaffolds of the present invention include, but are not limited to extracellular matrix proteins, cell adhesion proteins, growth factors, cytokines, hormones, proteases and protease substrates. Thus, exemplary proteins include vascular endothelial-derived growth factor (VEGF), activin-A, retinoic acid, epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, erythropoietin, interleukins, tumor necrosis factors, interferons, colony stimulating factors, basic and acidic fibroblast growth factors, nerve growth factor (NGF) or muscle morphogenic factor (MMP). The particular growth factor employed should be appropriate to the desired cell activity. The regulatory effects of a large family of growth factors are well known to those skilled in the art.

Seeding of the cells on the scaffolds is also a critical step in the establishment of the vascularized cardiac tissue of the present invention. Since it has been observed that the initial distribution of cells within the scaffold after seeding is related to the cell densities subsequently achieved, methods of cell seeding require careful consideration. Thus, cells can be seeded in a scaffold by static loading, or, more preferably, by seeding in stirred flask bioreactors (scaffold is typically suspended from a solid support), in a rotating wall vessel, or using direct perfusion of the cells in medium in a bioreactor. Highest cell density throughout the scaffold is achieved by the latter (direct perfusion) technique. An exemplary seeding procedure is described in the Materials and Methods section herein below.

The cells may be seeded directly onto the scaffold, or alternatively, the cells may be mixed with a gel which is then absorbed onto the interior and exterior surfaces of the scaffold and which may fill some of the pores of the scaffold. Capillary forces will retain the gel on the scaffold before hardening, or the gel may be allowed to harden on the scaffold to become more self-supporting. Alternatively, the cells may be combined with a cell support substrate in the form of a gel optionally including extracellular matrix components. An exemplary gel is Matrigel™, from Becton-Dickinson. Matrigen™ is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Matrigel™ also contains growth factors, matrix metalloproteinases (MMPs [collagenases]), and other proteinases (plasminogen activators [PAs]) (Mackay, A. R., et al., BioTechniques 15:1048, 1993). The matrix also includes several undefined compounds (Kleinman, H. K., et al., Biochem. 25:312, 1986; McGuire, P. G. and Seeds, N. W., J. Cell. Biochem. 40:215, 1989), but it does not contain any detectable levels of tissue inhibitors of metalloproteinases (TIMPs) (Mackay, A. R., et al., BioTechniques 15:1048, 1993). Alternatively, the gel may be growth-factor reduced Matrigel, produced by removing most of the growth factors from the gel (see Taub, et al., Proc. Natl. Acad. Sci. USA (1990); 87 (10:4002-6). In another embodiment, the gel may be a collagen I gel, alginate, or agar. Such a gel may also include other extracellular matrix components, such as glycosaminoglycans, fibrin, fibronectin, proteoglycans, and glycoproteins. The gel may also include basement membrane components such as collagen IV and laminin. Enzymes such as proteinases and collagenases may be added to the gel, as may cell response modifiers such as growth factors and chemotactic agents.

As mentioned above, the method of the present invention is effected by co-seeding cardiomyocytes and endothelial cells on a scaffold under conditions which allow the formation of at least one 3D endothelial structure within the scaffold

The present inventors have shown that one of these conditions comprises seeding the above mentioned cells in a medium which supports both endothelial tube structures as well as cardiac tissue survival. According to another embodiment, the medium also allows further differentiation of partially differentiated cardiomyocytes. An exemplary medium with these attributes is one which comprises a 50% endothelial medium (e.g. EGM2, Cambrex) and a 50% embryonic stem cell medium.

Another of these conditions comprises culturing for a sufficient time following initial seeding on the scaffold for the formation of a 3D endothelial structure. The present inventors have shown that a period of at least 1 week, more preferably 10 days and even more preferably two weeks is required for the formation of such structures.

The present inventors have shown that promotion of 3D endothelial structures may also be enhanced by addition of fibroblast cells (e.g. human embryonic fibroblasts). Fibroblasts may be isolated from tissue according to methods known to those skilled in the art (e.g. obtained from E-13 ICR embryos) or purchased from cell culture laboratories such as Cambrex Biosciences or Cell Essentials. According to one embodiment, the fibroblast cells are co-seeded with the cardiomyocytes and endothelial cells. Accordingly, a pool of cardiomyocytes, endothelial cells and fibroblasts (in the presence or absence of an appropriate gel, as described herein above) may be generated and seeded onto the scaffold.

Compositions obtained according to the methods described herein above typically comprise a heterogenous population of cells, including, but not limited to cardiac cells, (e.g. cardiomyocytes), endothelial cells and fibroblast cells. The present inventors have shown that the compositions obtained according to the methods described herein also comprise smooth muscle cells.

Thus, according to another aspect of the present invention, there is provided an isolated composition of matter comprising vascularized cardiac tissue seeded on a scaffold. The vasculature of the vascularized cardiac tissue typically forms part of a 3D structure, such as a lumen. According to one embodiment, the vasculature comprises at least about 1% of the total tissue which is seeded on the scaffold. As demonstrated in the Examples section herein below, such a degree of vasculature is sufficient for survival of the cardiac tissue for at least two weeks following transplantation.

Since the compositions of the present invention comprise cardiac tissue that is capable of synchronous contractions and capable of responding to chronotropic agents, they may be used for treating a cardiac disorder which is associated with a defective or absent myocardium.

Thus, according to another aspect of the present invention there is provided a method of treating cardiac disorder associated with a defective or absent myocardium in a subject, the method comprising transplanting a therapeutically effective amount of the compositions of the present invention into the subject, thereby treating diabetes.

The method may be applied to repair cardiac tissue in a human subject having a cardiac disorder so as to thereby treat the disorder. The method can also be applied to repair cardiac tissue susceptible to be associated with future onset or development of a cardiac disorder so as to thereby inhibit such onset or development.

The present invention can be advantageously used to treat disorders associated with, for example, necrotic, apoptotic, damaged, dysfunctional or morphologically abnormal myocardium. Such disorders include, but are not limited to, ischemic heart disease, cardiac infarction, rheumatic heart disease, endocarditis, autoimmune cardiac disease, valvular heart disease, congenital heart disorders, cardiac rhythm disorders, impaired myocardial conductivity and cardiac insufficiency. Since the majority of cardiac diseases involve necrotic, apoptotic, damaged, dysfunctional or morphologically abnormal myocardium, and since the vascularized cardiac tissue of the present invention displays a highly differentiated, highly functional, and proliferating cardiomyocytic phenotype, the method of repairing cardiac tissue of the present invention can be used to treat the majority of instances of cardiac disorders.

According to one embodiment, the method according to this aspect of the present invention can be advantageously used to efficiently reverse, inhibit or prevent cardiac damage caused by ischemia resulting from myocardial infarction.

According to another embodiment, the method according to this aspect of the present invention can be used to treat cardiac disorders characterized by abnormal cardiac rhythm, such as, for example, cardiac arrhythmia.

As used herein the phrase “cardiac arrhythmia” refers to any variation from the normal rhythm of the heart beat, including, but not limited to, sinus arrhythmia, premature heat, heart block, atrial fibrillation, atrial flutter, pulsus alternans and paroxysmal tachycardia.

According to another embodiment, the method according to this aspect of the present invention can be used to treat impaired cardiac function resulting from tissue loss or dysfunction that occur at critical sites in the electrical conduction system of the heart, that may lead to inefficient rhythm initiation or impulse conduction resulting in abnormalities in heart rate.

The method according to this aspect of the present invention is effected by transplanting a therapeutically effective dose of the composition of the present invention to the heart of the subject, preferably by injection into the heart of the subject.

As used herein, “transplanting” refers to providing the scaffold supported cells of the present invention, using any suitable route.

As used herein, a therapeutically effective dose is an amount sufficient to effect a beneficial or desired clinical result, which dose could be administered in one or more administrations. According to one embodiment, a single administration is employed. The injection can be administered into various regions of the heart, depending on the type of cardiac tissue repair required. Intramyocardial administration is particularly advantageous for repairing cardiac tissue in a subject having a cardiac disorder characterized by cardiac arrhythmia, impaired, cardiac conducting tissue or myocardial ischemia.

Such transplantation directly into cardiac tissue ensures that the administered cells/tissues will not be lost due to the contracting movements of the heart.

The compositions of the present invention can be transplanted via transendocardial or transepicardial injection, depending on the type of cardiac tissue repair being effected, and the physiological context in which the cardiac repair is effected. This allows the administered cells or tissues to penetrate the protective layers surrounding membrane of the myocardium.

Preferably, a catheter-based approach is used to deliver a transendocardial injection. The use of a catheter precludes more invasive methods of delivery wherein the opening of the chest cavity would be necessitated.

In addition, as is described in Example 8 of the Examples section which follows, the positive and negative chronotropic responses to isoproterenol and carbamylcholine demonstrate the presence of functional adrenergic and cholinergic receptors, respectively, in the vascularized cardiac tissue of the present invention. Thus, the cultured cardiomyocytes of the present invention can be utilized to regulate the contraction rate of a heart en response to physiological or metabolic state of the recipient individual, thereby serving as a biological pacemaker.

In the case of repairing cardiac tissue in a subject having a cardiac disorder characterized by cardiac arrhythmia, electrophysiological mapping of the heart and/or inactivation of cardiac tissue by radiofrequency treatment may be advantageously performed in combination with administration of the cells and tissues of the present invention if needed.

To repair cardiac tissue damaged by ischemia, for example due to a cardiac infarct, the vascularized cardiac tissue of the present invention is preferably administered to the border area of the infarct. As one skilled in the art would be aware, the infarcted area is grossly visible, allowing such specific localization of application of therapeutic cells to be possible. The precise determination of an effective dose in this particular case may depend, for example, on the size of an infarct, and the time elapsed following onset of myocardial ischemia.

According to one embodiment, transplantation of the vascularized cardiac tissue of the present invention for repair of damaged myocardium is effected following sufficient reduction of inflammation of affected cardiac tissues and prior to formation of excessive scar tissue.

The present invention can be used to generate cardiomyocytic cells and tissues displaying a desired proliferative capacity, thus cells and tissues are preferably selected displaying a suitable proliferative capacity for administration, depending on the type of cardiac tissue repair being effected. Administration of highly proliferative cells may be particularly advantageous for reversing myocardial damage resulting from ischemia since, as previously described, it is the essential inability of normal adult cardiomyocytes to proliferate which causes the irreversibility of ischemia induced myocardial damage.

Since porcine models are widely considered to be excellent models for human therapeutic protocols and since such models have been widely employed and characterized, it is well within the grasp of the ordinarily skilled artisan to determine a therapeutically effective dose for a human based on the guidance provided herein, and on that provided by the extensive literature of the art.

Determination of an effective dose is typically effected based on factors individual to each subject, including, for example, weight, age, physiological status, medical history, and parameters related to the cardiac disorder, such as, for example, infarct size and elapsed time following onset of ischemia. One skilled in the art, specifically a cardiologist, would be able to determine the amount and number of cells comprised in the composition of the present invention that would constitute an effective dose, and the optimal mode of administration thereof without undue experimentation.

It will be recognized by the skilled practitioner that when administering non-syngeneic cells or tissues to a subject, there is routinely immune rejection of such cells or tissues by the subject. Thus, the method of the present invention may also comprise treating the subject with an immunosuppressive regimen, preferably prior to such administration, so as to inhibit such rejection. Immunosuppressive protocols for inhibiting allogeneic graft rejection, for example via administration of cyclosporin A, immunosuppressive antibodies, and the like are widespread and standard practice in the clinic.

Encapsulation techniques may also be performed in order to reduce the degree of immune rejection to non-autologous cells. Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules may be prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002;13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

The scaffold supported cells of the present invention may be transplanted to a human subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the scaffold supported cells of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Methods

Propagation of the hESC lines and in vitro cardiomyocyte differentiation: Pluripotent human embryonic stem cells (hESC) of the H9.2 clone (passage 30-60) were grown in the undifferentiated state on top of mouse embryonic fibroblast feeder layer as previously described [Xu C, et al., Circ Res. 2002;91:501-508; Levenberg S et al., Proc Natl Acad Sci USA. 2002;99:4391-6]. The culture medium consisted of 20% FBS (HyClone), 79% knockout DMEM supplemented with 1 mM L-glutamine, 0.1 mM mercaptoethanol, and 1% nonessential amino acids (all from Life Technologies).

To induce differentiation, the hESC were dispersed to small clumps using collagenase IV (1 mg/mL, Life Technologies). Then they were transferred to plastic petri-dishes and cultured in suspension for 7-10 days where they formed embryoid bodies (EBs). Hereupon the EBs were plated on gelatin-coated culture dishes. Spontaneously beating areas were noted in some of the EBs after 5-20 days of plating. The contracting areas within the EBs were microdissected with a curved 23G needle after 25-30 days of in vitro differentiation. The contracting areas were then dissociated into small cell clusters by incubation with 1 mg/ml of collagenase B (Roche) for 45 minutes as previously described [Xu C, et al., Circ Res. 2002; 91:501-508].

Human Umbilical Vein Endothelial Cells (HUVEC) (Clonetics, passage 3-6) were grown on regular tissue culture plates in EGM-2 medium supplemented with 2% FBS. Mouse embryonic fibroblasts were cultured in DMEM supplemented with 10% FBS.

Isolation of hESC derived endothelial cells: hESC derived CD31⁺ cells were isolated as described [Levenberg S et al., Proc Natl Acad Sci USA. 2002;99:4391-6]. Briefly, differentiating hEBs at days 13-15 were dissociated with 1% trypsin/EDTA (Beit Haemek, Israel) and incubated for 30 min with FITC-labeled anti CD-31 antibodies (BD Pharmingen) on ice. Fluorescent-labeled cells were isolated by using FACSVantge flow cytometry cell sorter (Becton Dickinson) and plated on 1% gelatin-coated plates with endothelial cell growth medium (Clonetics). Cells were passaged by using 0.025% trypsin EDTA (Cambrex Biosciences) and cultured in endothielial cell medium consisting of EGM-2 (Cambrex Biosciences).

Scaffolds: Porous sponges composed of 50% poly-1-lactic acid (PLLA) (Polysciences) and 50% polylactic-glycolic acid (PLGA) (Boehringer-Ingelheim) were fabricated as previously described [Levenberg, et al., Proc Natl Acad Sci USA. 2003;100:12741-12746] with pore sizes of 212-600 μm and 93% porosity. Briefly, PLLA and PLGA 1:1 were dissolved in chloroform to yield a solution of 5% polymer; 0.24 ml of this solution was loaded into molds packed with 0.4 g of sodium chloride particles. The solvent was allowed to evaporate, and the sponges were subsequently immersed for 8 hrs in distilled water (changed every hour) to leach the salt and create an interconnected pore structure. The sponges were sliced to squares at a volume of ˜9 mm³ (3 mm*3 mm*1 mm). Degradation time of the composed sponges is ˜6 months. For seeding, the desired number of cells were pooled and resuspended in 8-10 μl of a 1:1 mixture of culture medium and growth factor-reduced Matrigel (BD Biosciences). The suspension was allowed to absorb into the sponges, after which they were incubated for 20 minutes at 37° C. allowing solidification of the gel. Culture medium was then added. The sponges were detached from the bottom of the plate and incubated at 37° C. on a XYZ shaker. Every other day the medium was changed. It consisted of 50% EGM-2 medium and 50% standard ES cell culturing medium supplemented with 1% penicillin/streptomycin. Following two weeks of cell culture, scaffolds were fixed in 10% formalin and subsequently embedded in paraffin for sectioning.

Immuno staining: Immuno-staining of 5 μm sections was carried out using the Biocare Medical Universal HRP-DAB kit (Biocare Medical) according to manufacturer's instructions, with prior heat treatment at 95° C. for 20 minutes in ReVeal buffer (Biocare Medical) for epitope recovery. For stainings conducted with anti-vWF antibody deparaffinization and trypsin treatment were conducted for epitope recovery. Primary antibodies used were: monoclonal anti-human: CD31 (1:20); polyclonal rabbit anti-vWF (1:200); monoclonal anti Ki-67 MIB-I (1:30) and anti smooth muscle actin (1:50) (all from Dako); mouse anti-α-sarcomeric alpha actinin (1:100) (Sigma), polyclonal rabbit anti-connexin 43 (1:40), mouse anti troponin I (1:200) (Chemicon). For Immunofluorescent stainings, Secondary antibodies were: Cy3 and Cy2-conjugated anti-mouse IgG (1:100) (Jackson Immunoresearch laboratory, PA); Cy2-conjugated anti rabbit IgG (1:100); AlexaFluor 488 conjugated anti mouse IgG (1:100) (Molecular Probes) or by using Cy3 conjugated anti mouse IgG2b (1:100) (for double monoclonal antibody immunostaining of troponin I). Nuclei were counterstained using DAPI (Sigma). Fluorescence microscopy was performed using a Zeiss microscope (Axiovert 200M) and a CCD Camera (Axiocam, MRc5).

Endothelial cell, lumen area and lumen number density quantification: Microscopic pictures were randomly taken at a magnification of 10× and an imaging analysis software (Axiovision 3.1, Carl Zeiss) was used to determine the area of endothelial cells (based on anti-vWF and anti-CD-31 immunostainings), the area of vessels and the lumen formed by the endothelial cells and the total sample area. The number of structures was manually counted. p values were calculated using Student's t-test.

Cell viability assay: To test whether the addition of embryonic fibroblasts (EmF) results in improved endothelial cell survival, the viability of endothelial cells were evaluated following 1 hr, 24 hrs, 72 hrs and 1 week. Prior to cell molding, HUVEC cells were labeled with DAPI (1 μg/ml) for 45 minutes at 37° C. To assess cell viability scaffolds were loaded with Calcein AM (1 μM) and Ethidium homodimer-1 (4 μM) (Live/Dead® Viability/Cytotoxicity kit for mammalian cells, Molecular Probes) for 50 minutes at 37° C. on top of a 3D XYZ shaker. Following dye loading scaffolds were washed with PBS (×3) and then dissected to small pieces. To allow cell dissociation from scaffolds, sliced scaffold pieces were incubated with Trypsin-EDTA ×2 (Beit Haemek, Israel) for 8 minutes at 37° C. Pictures of dispersed single cells (isolated using 100 μm single cell filter) were taken in ×20 magnification using the inverted fluorescence microscope and CCD Camera described above. The percentage of cells stained positive with DAPI and Calcein AM or ethidium-1 homodimer was calculated.

Transmission Electron Microscopy: Scaffolds were fixed in 3% paraformaldehyde, 2% glutaraldehyde and 5 mM CaCl₂ in 0.1 M cacodylate buffer (pH 7.2) for 90 minutes and room temperature followed by overnight incubation at 4° C. Following the initial fixation, scaffolds were post fixed 1% OₛO₄ in 0.1 M cacodylate buffer, 5 mM CaCl₂, 0.5% Potassium dichromate and 0.5% Potassium hexacyonaferrate for 1 h. Scaffolds were stained with 2% aqueous uranyl acetate followed by ethanol dehydration. The scaffolds were then embedded in Epon 812 (Electron Microscopy Sciences). Sections were cut using LEICA Ultracut UCT microtome at a thickness of 70 nm with a diamond knife (Diatome, Biel, Switzerland). Sections were examined with a Tecnai 12 (FEI Company, Eindhoven) transmission electron microscope at an accelerating voltage of 120 kV. Pictures were taken with CCD camera (MegaView III, Soft Imaging System, Germany).

RT-PCR Studies: For semi quantitative RT-PCR and real time PCR analysis, scaffolds were frozen in liquid nitrogen 2 weeks following cell molding. Prior to RNA isolation, scaffolds were incubated with Trypsin×10 (Beit Haemek, Israel) for 8 minutes in the presence of 1 U/μL Rnase inhibitor (RNAsin (Promega, U.S.A.)) to allow cell dissociation. RNA was isolated from the dispersed cells using the High pure RNA isolation kit (Roche). Reverse transcription of the isolated RNA into cDNA was conducted using Reverse-iT 1^{st} Strand Synthesis Kit (ABgene) according to manufacture's instructions. PCR for the various genes was performed using primers and conditions detailed in Table 1. Briefly, each RT-PCR included 30 secs at 95° C., 30 secs at 56° C. and 1 min at 72° C. using Red Load Taq Master Mix (Larova, Germany). 20 ng of cDNA template was used from each scaffold sample.

Taq-man real time PCR studies were performed using assay on demand primers and probes (Applied-Biosystem) in 96-well optical plates in triplicates. The assay on demand primers used were: Troponin I (Hs00165957_ml), MHC (Hs00411908_ml), nkx2.5 (Hs00231763_ml) and ANF (Hs00383231_ml). Samples were cycled for 45 times using an ABI 7700 Sequence Detector (Applied Biosystems). ABI 7700 cycle conditions were as follows: 2 minutes at 50° C., 10 minutes at 95° C. followed by 45 cycles of 15 seconds at 95° C. and 1 minute at 60° C. C_{T} was calculated under default settings for real-time sequence detection software (Applied Biosystems).

Table 1 herein below lists the primers and sequences thereof used in the above described experiments.

TABLE 1

| | | | | |
|---|---|---|---|---|
| Gene | Species | cycles | Forward | Reverse |

| | | | | |
|---|---|---|---|---|
| hANF | Human | 40 | GAACCAGAGGGGAGAGACAGAG | CCCTCAGCTTGCTTTTTAGGAG |
| | | | SEQ ID NO:1 | SEQ ID NO:2 |
| MLC-2V | Human | 40 | TATTGGAACATGGCCTCTGGAT | GGTGCTGAAGGCTGATTACGTT |
| | | | SEQ ID NO:3 | SEQ ID NO:4 |
| Nkx2-5 | Human | 36 | CTTCAAGCCAGAGGCCTACG | CCGCCTCTGTCTTCTTCAGC |
| | | | SEQ ID NO:5 | SEQ ID NO:6 |
| MEF2C | Human | 36 | GAACAATCCCGGTGTGTCAGGA | CACCCAGTGGCAGCCTTTTACA |
| | | | SEQ ID NO:7 | SEQ ID NO:8 |
| MHC | Human | 36 | GTCATTGCTGAAACCGAGAATG | GCAAAGTACTGGATGACACGCT |
| | | | SEQ ID NO:9 | SEQ ID NO:10 |
| Cardiac | Human | 36 | GGAGTTATGGTGGGTATGGGTC | AGTGGTGACAAAGGAGTAGCCA |
| α-actin | | | SEQ ID NO:11 | SEQ ID NO:12 |
| Skeletal | Human | 36 | CGAGCCGAGAGTAGCAGTTG | CAGTTGGTGATGATGCCGTGC |
| α-actin | | | SEQ ID NO:13 | SEQ ID NO:14 |
| bFGF | Human | 36 | CTCATGCCCATATTCCCTGCACT | ACCAGGTTGGTCTTGAACTCCTGG |
| | | | SEQ ID NO:15 | SEQ ID NO:16 |
| GAPDH | Human | 30 | AGCCACATCGCTCAGACACC | GTACTCAGCGGCCAGCATCG |
| | | | SEQ ID NO:17 | SEQ ID NO:18 |
| AngI | Human | 36 | AGCGAATGGAAGCCCTTACA | CTCATCGAAGTGGACCGGCA |
| | | | SEQ ID NO:19 | SEQ ID NO:20 |
| AngI | Mouse | 36 | AGCGAATGGAAGCCCTTACA | CTCATCGAAGTGGACCGGCA |
| | | | SEQ ID NO:21 | SEQ ID NO:22 |
| PDGF-B | Mouse | 36 | GCAATAACCGCAATGTGCAATGCC | CGCCTTGTCATGGGTGTGCTTAAA |
| | | | SEQ ID NO:23 | SEQ ID NO:24 |
| VEGF | Mouse | 36 | CCTCCGAAACCATGAACTTTCTGCTC | CAGCCTGGCTCACCGCCTTGGCTT |
| | | | SEQ ID NO:25 | SEQ ID NO:26 |

Laser scanning confocal Ca-imaging: Scaffolds were loaded with fluo-4 Ca AM (Molecular Probes) indicator to visualize free Ca⁺² levels. To this purpose, 5 mM stock solution of fluo-4 AM in DMSO was, diluted in standard hESC medium, at dilution of 1:1000 giving a final concentration of 5 μM. The non-ionic detergent Pluronic F-127 (Molecular Probes) was used to assist dispersion of the nonpolar fluo-4 AM ester in the aqueous media. Scaffolds were incubated for 45 minutes at 37° C. Following incubation the scaffolds were washed (×3) with indicator-free control tyrode solution removing any dye non-specifically associated with the cell surface. Afterwards the scaffolds were further incubated for 15 minutes allowing complete de-esterification of intracellular AM esters (according to manufacture's instructions).

Intracellular calcium transients were imaged with a confocal imaging system (Olympus Fluoview) mounted on an upright BX51WI Olympus microscope equipped with a 60× (0.9 n.a.; Olympus) water objective. In addition the mild uncoupler 1-Heptanol (1 mM) was applied to evaluate whether electrical impulse propagation

Statistical Analysis: All results were expressed as mean±SEM. When comparing more than two groups, ANOVA was used followed by a post-hoc bonferoni's. Student's t-test or Mann whitney rank sum test was used to compare between two groups. A p value of 0.05 or less was judged to be statistically significant.

### Example 1

### Generation of the Engineered Human Cardiac Tissue

To explore the ability of establishing a three-dimensional supportive environment for generation of a vascularized cardiac tissue, PLLA (50%)/PLGA (50%) biodegradable scaffolds were used. The PLGA was selected to allow relatively fast degradation (˜3 weeks) to facilitate cellular ingrowths, whereas the PLLA was chosen to provide mechanical support for the three-dimensional structure. Three cell culture combinations were evaluated: (1) scaffolds seeded with human embryonic stem cells differentiated into cardiomyocytes (hESC-CM; 4*10⁵ cells) alone, (2) co-cultures comprised of hESC-CM (4*10⁵ cells) and HUVEC or hESC-derived EC (hESC-EC⁹) (4*10⁵ cells), and (3) a triple-cell culture comprised of hESC-CM, HUVEC or hESC-EC, supplemented with embryonic fibroblasts (EmF) (2*10⁵-4*10⁵ cells). The cells were seeded into the scaffolds together with matrigel to facilitate cell seeding and to keep the cells on the scaffolds.

The scaffolds were monitored microscopically for the appearance of spontaneous contraction every day following cell seeding. Synchronous contraction appeared initially after 4 days in the cardiomyocytes constructs (n=4). The regional contractions gradually spread until the entire scaffold was beating synchronously. A similar pattern of initiation of contraction (4-6 days) was also found in the scaffolds containing co-cultures of hESC-CM+HUVEC (n=6) and in the triple-cell culture of hESC-CM+HUVEC+EmF (n=5). The engineered cardiac tissue-constructs were observed for two weeks after which they were fixated and utilized for detailed histological examination. Histological analysis of the tissue-engineered constructs showed that the seeded cells lined both the inner and the outer surfaces of the scaffolds and that the hESC-CM could be identified in all scaffolds studied in all three groups (FIGS. 1A-C).

### Example 2

### Vascularization of the Engineered Human Cardiac Tissue

Scaffolds consisting of just hESC-CM contained only few vWF⁺ or CD-31⁺ cells (FIGS. 1A and 2A). The addition of HUVEC resulted in a significant increase in the quantity of endothelial cells (ECs) when compared to the scaffolds containing only hESC-CM (FIGS. 1B and 2B). Despite the increase in EC density, the EC did not organize into blood vessels and were mainly present as compact cell clusters (FIGS. 1B, 2B, and 3A).

The effects of adding embryonic fibroblasts (EmF) to the constructs containing hESC-CM and EC were examined. Examination of these tri-culture three-dimensional scaffolds revealed that the addition of EmF resulted in the generation of highly-vascularized engineered cardiac muscle (FIGS. 1C and 2C). Both immunohistochemical and immunofluorescence stainings demonstrated the organization of the EC into a condense network of vessels that was present within and in some cases also closely adjacent to the cardiac tissue (FIGS. 1C and 2C).

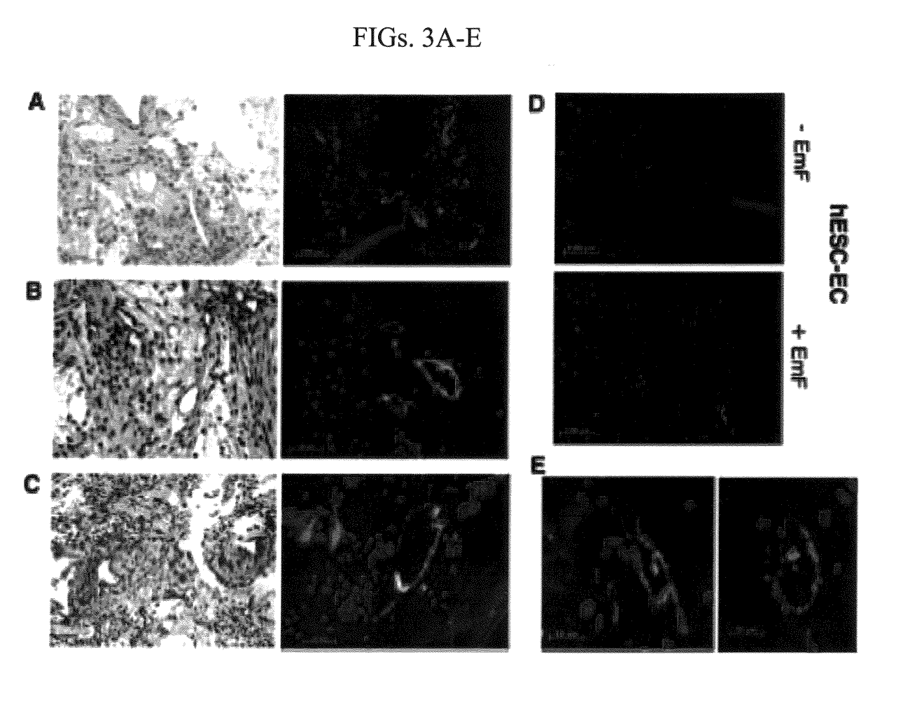

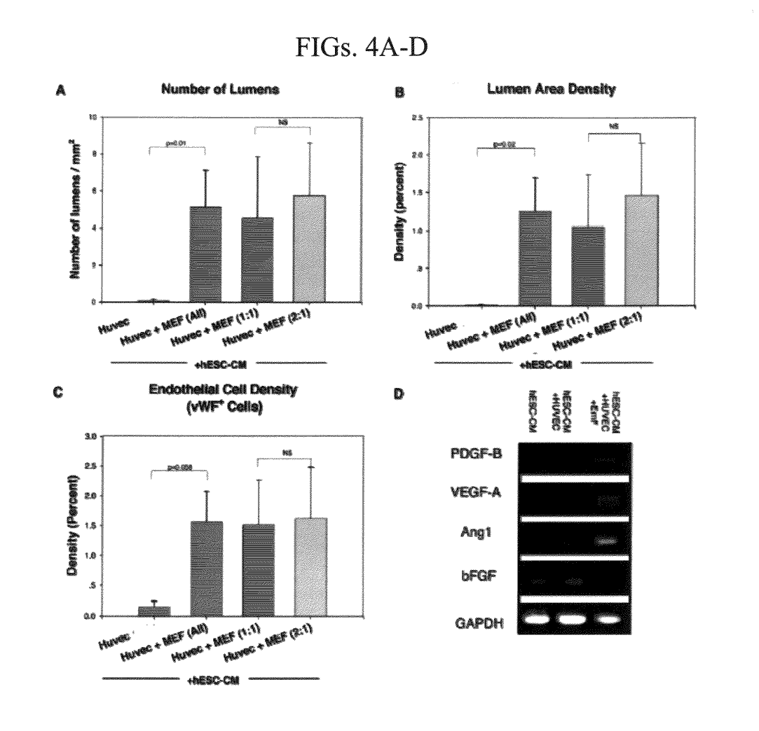

To further analyze the effects of the EmF on cardiac muscle vascularization quantitative immunostaining analysis was performed using anti-vWF antibodies. Three parameters of tissue vascularization and vessel organization were assessed: (1) the number of lumens per mm², (2) the lumen area density, and (3) the EC area density. Tri-culture scaffolds containing hESC-CM+HUVEC+EmF were characterized by a significantly higher number of vessels and displayed an increased lumen area density when compared to the co-cultures, which did not contain the EmF (FIGS. 3 and 4A-B). A higher EC density (stained positively for vWF) was also found in the tri-culture scaffolds (FIG. 4C). Comparison between the tri-culture scaffolds containing HUVEC:EmF at a ratio of 1:1 and 2:1 revealed no significant difference in the degree of vascularization based on the above mentioned parameters (FIGS. 3B, C and 4). The supporting effects of EmF on the organization of the EC into vessel networks were also sustained when the HUVEC were replaced with hESC-EC using similar cell ratios (FIG. 3D).

### Example 3

### Expression of Angiogenic and Vasculogenic Factors

To evaluate the expression of key angiogenic and vasculogenic factors in the three-dimensional vascularized cardiac tissue, the expression of vascular endothelial growth factor (VEGF-A), platelet derived growth factor (PDGF-B), Angiopoietin 1 (Ang1), and basic fibroblast growth factor (bFGF) was assessed at the mRNA level. Similar to the histological quantification of the vascularization process, the RT-PCR analysis revealed increased gene expression of the angiogenic factors VEGF-A, PDGF-B and Ang1 in the tri-culture cardiac tissue (upon addition of EmF) (FIG. 4D). An increase in the bFGF mRNA levels in the tri-culture was not noted.

A major factor known to contribute to EC organization is the presence of pericytes or smooth muscle cells. It was therefore assessed whether the EmF in the tri-cultures differentiated into smooth muscle cells. Immunostainings for α-smooth muscle actin (SMA) demonstrated the presence of SMA⁺ cells within the engineered cardiac tissue (FIG. 3E). In many cases, these SMA⁺ cells were demonstrated to integrate into the formed blood vessels and were localized adjacent to vWF+ cells (FIG. 3E).

### Example 4

### Temporal Assessment of EC Viability and Proliferation

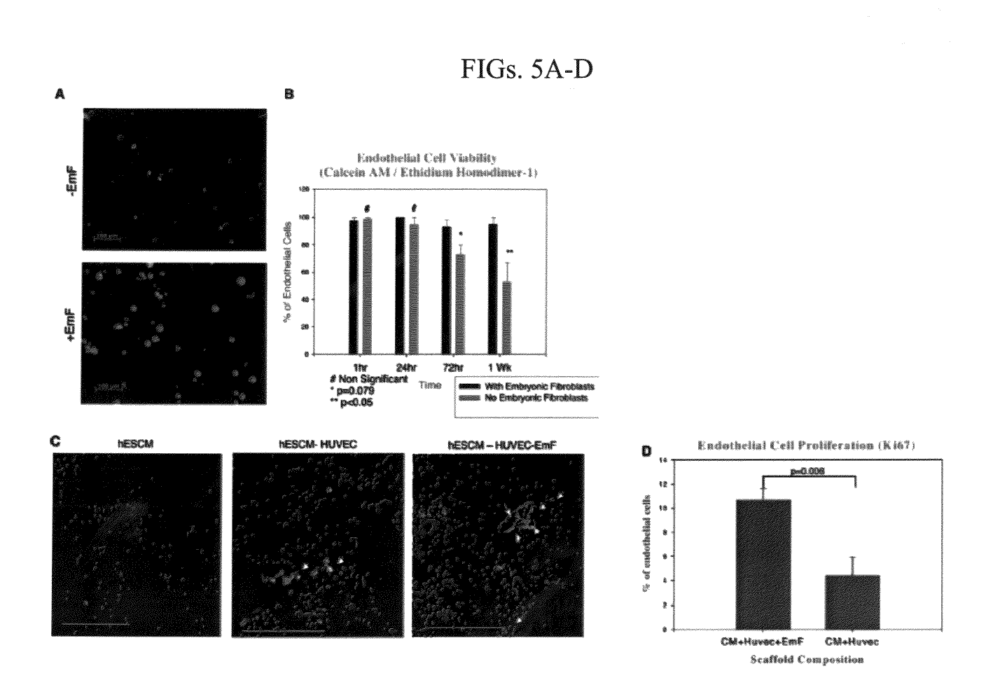

Since the presence of EmF affected not only the degree of EC organization but also the EC density, it was hypothesized that EmF may also influence EC viability and proliferation. Therefore, the degree of EC viability was assessed at several time points following cell seeding (1 hr, 24 hrs, 72 hrs, and 1 wk). The EC were pre-labeled with DAPI and cell viability was evaluated using Calcien AM (staining viable cells) and Ethidium Homodimer 1 (staining dead cells) (FIG. 5A). At 1 hr and 24 hrs following cell seeding, there were no significant differences in cell viability between the scaffolds with and without EmF. However, at 72 hours and 1 week, EC viability was significantly higher in scaffolds containing EmF (FIG. 5B).

An alternative explanation to the higher number of EC in the tri-culture compared to the co-culture scaffolds may be related to alteration of the proliferative capacity of the EC. To quantify this aspect, double immunostainings were performed for human Ki-67 (a marker for cycling cells) and vWF (FIG. 5C). It was found that the percentage of proliferating EC within the tri-culture (10.7±1%) was significantly higher (p<0.01) than those of the co-culture (4.4±1.5%) (FIG. 5D).

### Example 5

### Cardiomyocyte Structural Organization and Proliferation

The cardiomyocyte tissue within the scaffold was characterized. As can be seen in the immunohistochemistry images in FIGS. 1A-C, the cardiomyocytes were arranged in aggregates, some of which consisted of relatively small hESC-CM being isotropically arranged, while others were comprised of longitudinally oriented cell bundles containing more structurally mature cardiomyocytes. The latter areas were mainly located at the periphery of the scaffolds.

The unorganized, smaller, hESC-CM with higher nuclear to cytoplasmatic ratio (FIG. 6A arrows), usually denote a less mature stage of cardiomyocyte development [Snir M et al., Am J Physiol Heart Circ Physiol. 2003;285:H2355-H2363]. Double immunostaining studies with anti-troponin I and anti-human Ki-67 antibodies revealed that these areas of small cardiomyocytes contained many proliferating cardiomyocytes (FIG. 6A, arrows). In contrast, positively stained Ki-67 cardiomyocytes were rarely found in the more structurally organized areas and the cardiomyocytes in these regions were larger and displayed a more mature structural phenotype (FIG. 6A) p The effect of ECs in the engineered cardiac tissue was next assessed on the level of cardiomyocyte proliferation. Interestingly, as shown in FIGS. 6A-B, the percentage of the proliferating cardiomyocytes (Ki-67⁺) was significantly higher in the scaffolds containing the EC both in the presence and absence of EmF when compared with scaffolds containing only cardiomyocytes.

### Example 6

### Expression of Cardiac Differentiation Markers in the Engineered Cardiac Tissue

To assess the effect of the co/tri-culture system on the differentiation and maturation of the hESC-CM semi-quantitative RT-PCR was carried out (FIG. 6C) and quantitative real-time RT-PCR (FIG. 6D) studies evaluating both markers of early-immature cardiomyocytes (atrial natriuretic factor (ANF), Nkx2.5, MEF2C and α-skeletal actin) and markers of more mature, differentiated, cardiomyocytes (Myosin light chain 2V, α myosin light chain, α-cardiac actin and Troponin I). Gene expression analysis revealed up-regulation in the expression of markers of cardiomyocyte maturation such as MLC-2V, Troponin I and α cardiac actin. However, the levels of α myosin heavy chain were only mildly effected by the cell combination used (FIG. 6C,D). Surprisingly, the up-regulation of cardiomyocyte maturation markers was not accompanied by down-regulation of gene markers of early and immature cardiomyocytes. One possible explanation to the latter phenomenon may be the presence of areas containing highly dividing immature cardiomyocytes also within the co- and tri-cultures scaffolds (FIG. 6A arrows).

### Example 7

### Ultrastructural Characterization of the Engineered Cardiac Tissue

Transmission electron microscopy of the scaffolds demonstrated the presence of cardiomyocytes in both the early-immature and more mature stages of development. The immature cardiomyocytes were fewer and were characterized by the presence of relatively disorganized myofibrils (FIG. 7A) which in some cases were associated with a distinct electron dense material (the developing Z-bodies). In contrast, myofibrils were more abundant in the relatively mature cells. They were organized in similar directions, and were confined to parallel Z bands forming the typical sarcomeric pattern (FIG. 7B). The cardiomyocytes contained mitochondria that were packed around the sarcomeres (FIG. 7B). Beyond the presence of mitochondria and sarcomeric organization, the hallmarks of more mature cardiomyocytes are the presence of T-tubules and sarcoplasmic reticulum and the formation of gap junctions. In some cells, the presence of developing T-tubules associated with sarcoplasmic reticulum (Dyads) could be noted (FIG. 7B), which were located around the sarcomeric structures. In addition, the presence of specialized junctional structures responsible for electromechanical coupling between neighboring cardiomyocytes could also be detected. These included the presence of intercalated discs containing desmosomes and gap junctions (FIG. 7C, D). Similarly, immunofluorescent staining demonstrated the formation of gap junctions comprised of connexin 43 between the human cardiomyocytes (FIG. 7E, F).

### Example 8

### Impulse Propagation

The engineered cardiac tissue demonstrated spontaneous synchronous contractions of the cardiomyocytes within and between scaffold pores. Coupling of cardiomyocyte contraction and electrical excitation is known to be mediated via trans-membrane Ca⁺² influx and intracellular Ca⁺² release. To evaluate the presence of synchronous Ca⁺² transients within the engineered cardiac tissue, laser confocal Ca⁺² imaging studies were carried out utilizing the free Ca⁺² binding dye, Fluo-4. FIG. 8A depicts a typical line scan image through 6 cells. Note the synchronous surges of intracellular Ca⁺² levels within the contracting hESC-CM.

Since the ultrastructural characterization clearly indicated the presence of gap junctions between adjacent hESC-CM (FIG. 7D), the present inventors sought to determine whether these formed gap junctions mediate impulse conduction between the hESC-CM and therefore allow synchronous contraction. The gap junction uncoupler, 1-heptanol (1 mM), was applied to the spontaneously contracting engineered cardiac tissue. As expected, administration of 1-heptanol resulted in complete inhibition of impulse propagation as identified by the calcium imaging studies (FIG. 8B). The beating frequency of the spontaneously contracting cardiac tissue was evaluated following application of different pharmacological agents. Appropriate positive and negative chronotropic responses were observed following application of the β-agonist, isoproterenol (1 μM) and the muscarinic agonist—carbamylcholine (1 μM). Thus, isoproterenol increased the beating frequency of the engineered cardiac issue from 1.6±0.3 Hz to 2.1±0.3 Hz (p<0.05, n=4), while carbamylcholine decreased the beating frequency from 1.7±0.2 Hz to 1.4±0.1 Hz (p=0.055, n=4).

Discussion

The present study describes, for the first time, the generation of a three-dimensional, engineered, vascularized, human cardiac tissue that is based on the use of hESC. Ultrastructural characterization of the engineered cardiac tissue revealed the presence of differentiating cardiomyocytes with a typical sarcomeric pattern, formation of gap-junctions and dyads containing T-tubules and sarcoplasmic reticulum. Calcium imaging studies demonstrated that the generated cardiac tissue contracted spontaneously and synchronously with gap junctions mediating impulse propagation between the beating cardiomyocytes. The three-dimensional tissue also responded to both positive and negative chronotropic agents. Importantly, vascularization of the cardiac tissue was strongly promoted by the addition of HUVEC or hESC-EC and EmF. This was manifested by increased density of vessels within the cardiac tissue accompanied by the generation of stabilized vessels containing smooth muscle cells. Gene expression analysis revealed that upregulation of key angiogenic and vasculogenic markers occurred in the tri-culture engineered tissue. Intriguingly, it was found that the addition of EmF significantly augmented EC density within the engineered tissue possibly through the augmentation of both EC survival and proliferation capacity. Similarly, the presence of EC significantly increased the proliferative capacity of the cardiomyocytes in the co/tri-culture conditions. Finally, the vascularization of the cardiac vessel network within the engineered tissue resulted in upregulation of both early and late markers of cardiomyocyte differentiation and maturation.

Replacement of defective myocardial areas by functional cardiomyocytes undoubtedly depends on the ability of the grafted tissue to survive within the hostile ischemic environment. Previous studies, utilizing various cell sources, revealed that transplantation of single cells results in significant cell death. An alternative approach to transplantation of single cells may be replacement of diseased myocardial areas by in-vitro designed three-dimensional engineered cardiac tissue. Preformed cardiac matrices allow the delivery of longitudinally aligned cardiomyocytes forming a synchronously-contracting and well-coupled muscle network. However, a major limitation of this approach is the maximal size of the constructed tissue. This is mainly due to the high metabolic demands, inherent intolerance of anaerobic metabolism and the compact nature of the cardiac muscle strands. Consequentially, the maximum size of engineered cardiac muscle is confined by the maximum diffusion distance of oxygen and nutrients (˜100 μm).

Thus, construction of clinically-relevant cardiac tissues must allow full thickness perfusion of the preformed cardiac muscle. This issue is even of greater importance when considering the scarce vascularization of the myocardial scar. The spontaneous development of primitive capillaries within cardiac tissue-constructs that were reported in studies using primary cultures of neonatal rat ventricular cardiomyocytes [Zimmerman W H et al., Circ Res. 2002;90:223-230] probably stems from the mixed population of cells present in the rat ventricle. This vasculature provided a partial solution to this key limiting problem, and probably promoted the survival of implanted cardiac scaffolds in subsequent experiments [Zimmerman W H et al., Biomaterials. 2004;25:1639-1647; Zimmerman W H et al., Nat Med. 2006;12:452-458]. However, construction of an engineered cardiac-tissue from the potential clinically-relevant cell source of hESC-CM did not result in the generation of significant capillary network when used alone.

The tri-culture system of the present invention resulted in the generation of highly vascularized three-dimensional cardiac tissue accompanied by the integration of smooth muscle cells to the newly formed capillaries.

The positive effect of the embryonic fibroblasts on tissue vascularization was evident by an increase in the density of the endothelial cells. Two possible mechanisms were suggested to explain this finding. First, temporal assessment of endothelial cell viability revealed that the presence of embryonic fibroblasts inhibited endothelial cell death. The second mechanism for the increased endothelial cell density in the tri-culture is the significant increase in degree of EC proliferation. This effect may also be attributed to endothelial mitogenic factors such as VEGF-A, PDGF-B and Ang-1 (up-regulated in the tri-cultures and known to be secreted from embryonic fibroblasts).

An important finding of this study was the relatively high-degree of cardiomyocyte proliferation when endothelial cells were added when compared to scaffolds containing only hESC-CM (in which the proliferation rate was similar to that previously reported in similar-stage cultured EBs [Snir M et al., Am J Physiol Heart Circ Physiol. 2003;285:H2355-H2363). Since the quantity of cycling cardiomyocyte was augmented in both the co- and tri-culture conditions, it may be speculated that cell-cycle activation was the result of the interaction between hESC-CM and the EC.

### Example 9

### In vivo Transplantation of the Scaffolds of the Present Invention

Materials and Methods

Prior to surgical procedure, Sprague Dolly (SD) rats received an intramuscular injection of cyclosporine (2.5 mg/kg), solumedrol (3 mg/kg) and amoxicillin (30 mg/kg).
1. Rats were anesthetized with an intramuscular injection of a combination of ketamine (87 mg/kg) and xylazine (13 mg/kg). The chest was shaved and sterilized with iodine ointment.
2. Rats were intubated and mechanically ventilated with room air using a Harvard small-animal mechanical respirator, set at a tidal volume of 1 ml/100 g body weight and at a rate of 100 strokes/minute.
3. An oblique incision in the skin, between left axilla and the xiphoid, was performed and the chest muscles were revealed.
4. Major and minor pictoralis muscles were separated using fine scissors, until clear observation of the ribs.
5. The second left intercostal space was opened by a left thoracotomy. The ribs were gently spread using a small-sized retractor.
6. The pericardium was dissected, and, if necessary, the thymus was cut partially, in order to reveal the left atrium and to observe the left anterior descending artery.
7. A single stitch was placed through the myocardium at a depth slightly greater than the perceived level of the left anterior descending artery (LAD) of the left coronary artery while taking care not to enter the ventricular chamber. The sutures were tightened permanently using 6-0 prolene suture, to occlude the LAD. Paling of the left ventricle and prominence of the veins on the left ventricular wall indicated that the occlusion was successful.
8. The incision in the left intercostal space was closed using a 4-0 vicryl suture.
9. The skin was closed using a 3-0 silk suture.
10. The rat was disconnected from the ventilator repetitively and monitored for appearance of spontaneous breathing.
11. Once the rat was breathing spontaneously, with no signs of dyspnea or tachypnea, the intubation tube was removed and the rat was allowed to recover.

The rats receive daily doses of intramuscular injection of cyclosporine (2.5 mg/kg) and solumedrol (3 mg/kg) until the scaffold transplantation procedure.
Transplantation of the engineered cardiac tissue: The transplantation surgery was performed one week following myocardial infraction (Scaffolds were also sutured to healthy rat hearts, as part of the experiment).
1. The rats were anesthetized, intubated and mechanically ventilated as described earlier.
2. The adhesions were separated using fine scissors.
3. A left thoracotomy was done in the third or fourth left intercostal space, until the heart was revealed and the infracted zone visualized.
4. The transplantation procedure: Following two weeks of in-vitro cell-scaffold culture, the cell-seeded scaffolds were sutured to the infracted zone of an acutely injured heart by a 6-0 prolene suture.
5. The chest and skin were closed and the rat extubated as discussed earlier. The rats were allowed to recover.

Echocardiographic assessment: The rats underwent a baseline echocardiographic examination one week after the myocardial infarction surgery and one month following it. Echocardiography was performed after induction of anesthesia using ketamine (40 mg/kg) and xylazine (6 mg/kg).

Rat sacrificing: One month (for the infraction experiments) or two weeks (for the healthy hearts experiments) after the transplantation surgery the rats were anesthetized with a large dose of ketamine and xylazine, the chest opened and the hearts dissected.

CM-DiO labeling: CM-DiO (Molecular probes, Inc. Eugene, Oreg., USA) is a cell tracker that is retained in cells throughout fixation, permeabilization, and paraffin embedding procedures. A working solution of CM-DiO was prepared (concentration of 1.5 μM (molecular weigh of CM-DiO≈1000). The cells were incubated with the working solution for 5 minutes at 37° C., and then for an additional 15 minutes at 4° C. The cells were then washed with PBS and resuspended in fresh medium.

Microspheres injection: Fluorescent microspheres for blood flow determination (FluoSpheres, Molecular Probes) were used for assessment of perfusion of the transplanted scaffold in vivo. Prior to euthanization, and after occlusion of the great vessels of the heart, 10×10⁵ fluorescent microspheres beads (1 ml suspension, 10 μm diameter, green fluorescence) were injected into the left ventricle using 29G syringe, at a rate of 1 ml/min. The animal was then euthanized and the heart dissected. For the procedure evaluating the scaffold fluorescence intensity, the scaffold was detached from the heart and digested. Digestion of the scaffold was accomplished using chloroform, while digestion of the tissue within the scaffold was accomplished in 10 ml of 4 M potassium hydroxide (KOH) for 24 hours at 60° C. After complete digestion of the tissue, the microspheres were collected by centrifuging at 2000 g for 20 minutes and washing with 10 ml deionized water. Finally, microspheres were dissolved in Xylene and the intensity of fluorescence was determined by using a fluorescence spectrometer.

For evaluating microspheres in paraffin sections (for quantitative histology), the hearts were proceeded for regular paraffin embedding procedure without any digestion. The number of microspheres per scaffold area were counted and normalized to number of microspheres per heart cross section area.

Lectin injection: Lectin HPA (Helix pomatia agglutinin) conjugated to Alexa flour 488 (Molecular Probes) was injected to the left ventricle prior to euthanization, and after occlusion of the great vessels of the heart.

Results

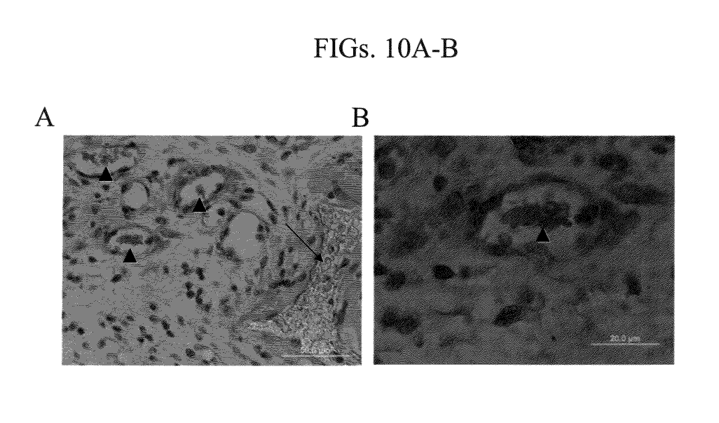

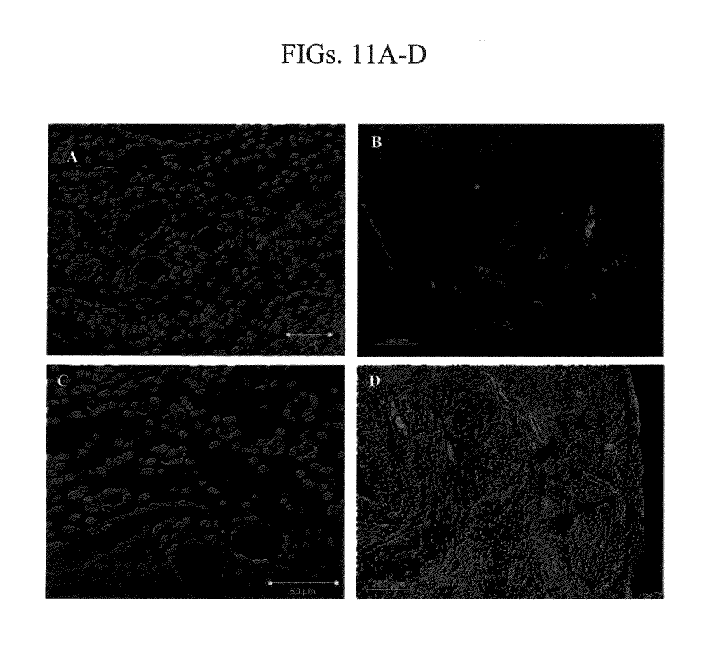

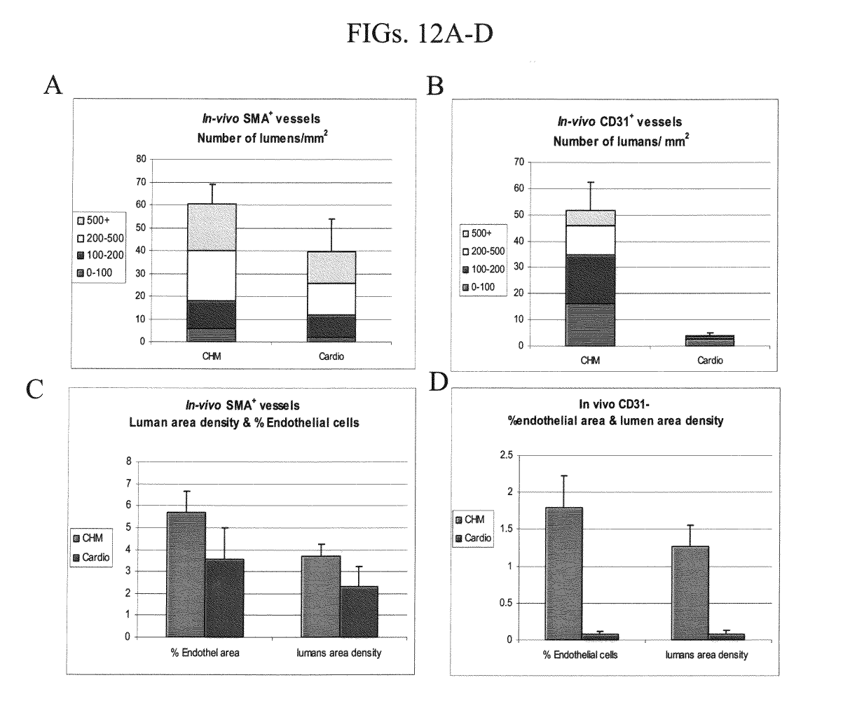

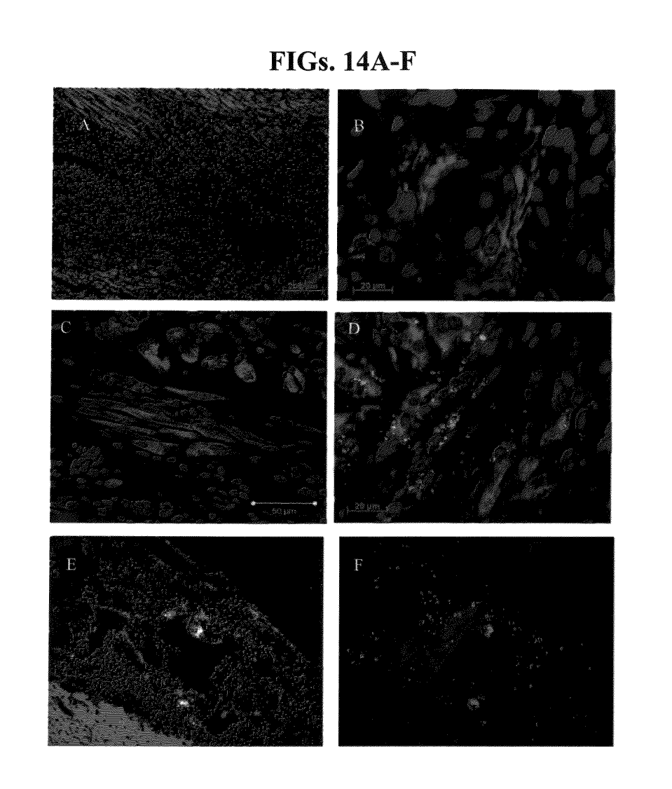

Transplantation of the engineered cardiac tissue to the healthy rat heart resulted in tight attachment of the scaffold to the host myocardium (FIGS. 9A-B) and in the generation of intense neo-vascularization (confirmed by H&E staining, SMA and VWF antibody staining) (FIGS. 10A-B and 11A-D). Human vessels were also evidenced in the graft area (positively stained with human specific CD31 antibody) (FIGS. 11A, B). The amount of vessels originating from the host (noe-vascularization) and the human vessels were quantified (see FIGS. 12A-D) in the two experimented groups. The amount of blood vessels in the tri-culture scaffold was higher compared to the cardiac-only scaffold (higher number of blood vessels, higher lumen area density and higher endothelial percentage). Moreover, functional vessels were present in the scaffold area (evidenced by the appearance of red blood cells, Lectin perfusion and Microspheres injection) (FIGS. 10A-B and 13A-D). Survival and maturation of the human embryonic stem cells derived cardiomyocytes (hES-CM) within the graft area was confirmed by DiO tracing and immunostaining to Troponin I antibody, revealing the presence of elongated and well-aligned striated cardiomyocytes (FIGS. 14A-F).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An isolated composition of matter comprising a heterogeneous population of cells seeded on a scaffold, wherein said heterogeneous population of cells comprises cardiomyocytes, endothelial cells and fibroblast cells.

2. The isolated composition of matter of claim 1, wherein said heterogeneous population of cells are homogeneously distributed on said scaffold.

3. The isolated composition of matter of claim 1, wherein said scaffold is a porous scaffold.

4. The isolated composition of matter of claim 3, wherein a pore of said porous scaffold comprises a minimal average pore diameter of about 200 μm.

5. The isolated composition of matter of claim 3, wherein a pore of said porous scaffold comprises a maximal average pore diameter of about 800 μm.

6. The isolated composition of matter of claim 1, wherein said scaffold comprises poly(L-lactic acid) and poly(lactic acid-co-glycolic acid).

7. The isolated composition of matter of claim 1, wherein said scaffold comprises a 50:50 mixture of poly(L-lactic acid) and poly(lactic acid-co-glycolic acid).

8. The isolated composition of matter of claim 1, wherein said scaffold is biodegradable.

9. The isolated composition of matter of claim 1, wherein said scaffold is non-biodegradable.

10. The isolated composition of matter of claim 1, wherein said scaffold comprises a material selected from the group consisting of collagen-GAG, collagen, fibrin, PLA, PGA, PLA-PGA co-polymer, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, non-biodegradable polyurethane, polyurea, poly(ethylene vinyl acetate), polypropylene, polymethacrylate, polyethylene, polycarbonate and poly(ethylene oxide).

11. The isolated composition of matter of claim 1, wherein said scaffold is coated with a gel.

12. The isolated composition of matter of claim 11, wherein said gel is selected from the group consisting of a collagen gel, an alginate, an agar, a growth factor-reduced Matrigel, and MATRIGEL™.

13. The isolated composition of matter of claim 1, wherein said fibroblasts are human embryonic fibroblasts.

14. The isolated composition of matter of claim 1, wherein said cardiomyocytes are human cardiomyocytes.

15. The isolated composition of matter of claim 1, wherein said cardiomyocytes are derived from stem cells.

16. The isolated composition of matter of claim 15, wherein said stem cells are embryonic stem cells.

17. The isolated composition of matter of claim 15, wherein said stem cells are adult stem cells.

18. The isolated composition of matter of claim 1, wherein said endothelial cells are embryonic stem cell-derived endothelial cells or umbilical vein endothelial cells.

19. The isolated composition of matter of claim 18, wherein said embryonic stem cell-derived endothelial cells are mammalian stem cell derived endothelial cells.

20. The isolated composition of matter of claim 19, wherein said mammalian embryonic stem cell-derived endothelial cells are human stem cell derived endothelial cells.

21. The isolated composition of matter of claim 1, wherein said endothelial cells form part of a 3D endothelial structure.

22. The isolated composition of matter of claim 21, wherein said 3D endothelial structure comprises a lumen.

23. The isolated composition of matter of claim 1, wherein said scaffold is a non-fibrous scaffold.

24. An isolated composition of matter comprising a vascularized cardiac tissue attached to a scaffold.

25. The isolated composition of matter of claim 24, wherein said scaffold is a porous scaffold.

26. The isolated composition of matter of claim 24, wherein said scaffold is a non-fibrous scaffold.

27. The isolated composition of matter of claim 25, wherein a pore of said porous scaffold comprises a minimal average pore diameter of about 200 μm.

28. The isolated composition of matter of claim 25, wherein a pore of said porous scaffold comprises a maximal average pore diameter of about 800 μm.

29. The isolated composition of matter of claim 24, wherein said scaffold comprises poly(L-lactic acid) and poly(lactic acid-co-glycolic acid).

30. The isolated composition of matter of claim 24, wherein a vasculature of said vascularized cardiac tissue is sufficient for survival for at least two weeks of said cardiac tissue following transplantation.

31. The isolated composition of matter of claim 24, wherein said scaffold comprises a 50:50 mixture of poly(L-lactic acid) and poly(lactic acid-co-glycolic acid).

32. The isolated composition of matter of claim 24, wherein said scaffold is biodegradable.

33. The isolated composition of matter of claim 24, wherein said scaffold is non-biodegradable.

34. The isolated composition of matter of claim 24, wherein said scaffold comprises a material selected from the group consisting of collagen-GAG, collagen, fibrin, PLA, PGA, PLA-PGA co-polymer, poly(anhydride), poly(hydroxy acid), poly(ortho ester), poly(propylfumerate), poly(caprolactone), polyamide, polyamino acid, polyacetal, biodegradable polycyanoacrylate, biodegradable polyurethane and polysaccharide, polypyrrole, polyaniline, polythiophene, polystyrene, polyester, non-biodegradable polyurethane, polyurea, poly(ethylene vinyl acetate), polypropylene, polymethacrylate, polyethylene, polycarbonate and poly(ethylene oxide).

35. The isolated composition of matter of claim 24, wherein said scaffold is coated with a gel.

36. The isolated composition of matter of claim 35, wherein said gel is selected from the group consisting of a collagen gel, an alginate, an agar, a growth factor-reduced Matrigel, and MATRIGEL™.

37. The isolated composition of matter of claim 24, wherein said vascularized cardiac tissue comprises fibroblasts.

38. The isolated composition of matter of claim 37, wherein said fibroblasts are human embryonic fibroblasts.

39. The isolated composition of matter of claim 24, wherein said vascularized cardiac tissue comprises endothelial cells.

40. The isolated composition of matter of claim 39, wherein said endothelial cells are embryonic stem cell-derived endothelial cells or umbilical vein endothelial cells.

41. The isolated composition of matter of claim 40, wherein said embryonic stem cell-derived endothelial cells are mammalian embryonic stem cell derived endothelial cells.

42. The isolated composition of matter of claim 41, wherein said mammalian embryonic stem cell-derived endothelial cells are human stem cell derived endothelial cells.

43. The isolated composition of matter of claim 40, wherein said umbilical vein endothelial cells are human or mouse umbilical vein endothelial cells.

44. The isolated composition of matter of claim 39, wherein said endothelial cells are mammalian aortic endothelial cells.

45. The isolated composition of matter of claim 24, wherein said vascularized cardiac tissue comprises smooth muscle cells.

46. The isolated composition of matter of claim 24, wherein a vasculature of said vascularized cardiac tissue forms part of a 3D structure.

47. The isolated composition of matter of claim 46, wherein said 3D structure comprises a lumen.

48. The isolated composition of matter of claim 46, wherein a percent of said vasculature of said vascularized cardiac tissue is at least 1%.

49. The isolated composition of matter of claim 24, wherein the cardiac tissue is capable of spontaneous contraction.

50. The isolated composition of matter of claim 24, wherein the cardiac tissue is capable of responding to a chronotropic agent in a similar fashion to non-engineered cardiac tissue.

51. An isolated composition of matter comprising cardiomyocytes seeded on a porous scaffold, wherein a pore of said porous scaffold comprises a minimal average pore diameter of about 200 μm, said scaffold comprising a 50:50 mixture of poly(L-lactic acid) and poly(lactic acid-co-glycolic acid).

52. The isolated composition of matter of claim 51, further comprising endothelial cells seeded on said porous scaffold.

53. The isolated composition of matter of claim 51, further comprising fibroblasts seeded on said porous scaffold.

54. The isolated composition of matter of claim 51, further comprising smooth muscle cells seeded on said porous scaffold.

55. A method of ex vivo vascularizing cardiac tissue, the method comprising co-seeding cardiomyocytes and endothelial cells on a scaffold under conditions which allow the formation of at least one 3D endothelial structure within said scaffold, thereby ex vivo vascularizing the cardiac tissue.

56. The method of claim 55, wherein said conditions comprise an ex vivo culturing period of at least 1 week.

57. The method of claim 55, wherein said conditions comprise co-seeding fibroblast cells with said cardiomyocytes and said endothelial cells on said scaffold.

58. A method of treating a cardiac disorder associated with a defective or absent myocardium in a subject, comprising transplanting a therapeutically effective amount of the isolated composition of matter of claim 1 into the subject, thereby treating the cardiac disorder associated with a defective or absent myocardium.

59. A method of treating a cardiac disorder associated with a defective or absent myocardium in a subject, comprising transplanting a therapeutically effective amount of the isolated composition of matter of claim 24 into the subject, thereby treating the cardiac disorder associated with a defective or absent myocardium.

60. A method of treating a cardiac disorder associated with a defective or absent myocardium in a subject, comprising transplanting a therapeutically effective amount of the isolated composition of matter of claim 51 into the subject, thereby treating the cardiac disorder associated with a defective or absent myocardium.

61. A pharmaceutical composition comprising as an active agent the isolated composition of matter of claim 1, and a pharmaceutically acceptable carrier.

62. A pharmaceutical composition comprising as an active agent the isolated composition of matter of claim 24, and a pharmaceutically acceptable carrier.

63. A pharmaceutical composition comprising as an active agent the isolated composition of matter of claim 51, and a pharmaceutically acceptable carrier.

64. Use of the isolated composition of matter of claim 1 for the manufacture of a medicament identified for the treatment of a cardiac disorder associated with a defective or absent myocardium.

65. Use of the isolated composition of matter of claim 24 for the manufacture of a medicament identified for the treatment of a cardiac disorder associated with a defective or absent myocardium.

66. Use of the isolated composition of matter of claim 51 for the manufacture of a medicament identified for the treatment of a cardiac disorder associated with a defective or absent myocardium.
